(19) United States
(12) Patent Application Publication
(10) Pub. No.: US 2024/0241108 A1
(43) Pub. Date: Jul. 18, 2024

(54) LUNG ORGANOID MODEL AND METHOD OF USE

(71) Applicant: The Regents of the University of California, Oakland, CA (US)

(72) Inventors: Pradipta Ghosh, Oakland, CA (US); Soumita Das, Oakland, CA (US)

(21) Appl. No.: 18/563,147

(22) PCT Filed: Jun. 9, 2022
(86) PCT No.: PCT/US2022/032792

§ 371 Date: Nov. 21, 2023

Related U.S. Application Data
(60) Provisional application No. 63/208,522, filed on Jun. 9, 2021.

(51) Int. Cl. G01N 33/50; C12N 5/071

(52) U.S. Cl. CPC G01N 33/5082; C12N 5/0688; G01N 33/5091; C12N 2501/40; C12N 2501/415; C12N 2502/00; G01N 2500/10

(57) ABSTRACT

Compositions and methods useful as research tools for identifying agents effective in treating respiratory diseases and infections are disclosed herein. Methods include providing a composition comprising a human lung organoid model culture composition comprising human lung proximal airway epithelial cells and distal airway epithelial cells. Methods also include infecting the composition with a respiratory infectious agent. Methods further include administering a candidate therapeutic agent to the composition and determining whether a treatment effective response by the agent occurs in the composition.

### CROSS REFERENCE TO RELATED APPLICATIONS

This application claims priority to U.S. Provisional Application Ser. No. 63/208,522 filed on Jun. 9, 2021, the entire contents of which are incorporated herein by reference.

### TECHNICAL FIELD

The present invention relates to a method for modeling lung diseases using adult stem cell derived lung organoids.

### BACKGROUND

SARS-COV-2, the virus responsible for COVID-19, causes widespread inflammation and injury in the lungs, giving rise to diffuse alveolar damage (DAD)¹⁻⁵. Indicators of DAD include marked infection and viral burden leading to apoptosis of alveolar pneumocytes⁶, along with pulmonary edema^{7,8}. DAD leads to poor gas exchange and, ultimately, respiratory failure. Notably, respiratory failure appears to be a common final mechanism of death in patients with severe COVID-19 infection. However, it remains unclear how COVID-19 is capable of causing so much damage to a patient's lungs. A particular challenge is understanding the out-of-control immune reaction to the SARS-COV-2 virus many patients suffer, which is known as a cytokine storm. A cytokine storm has also been observed in many of the patients that died from severe COVID-19 infection. Although rapidly developed pre-clinical animal models have recapitulated some pathognomonic aspects of COVID-19 infection (e.g., induction of disease, transmission, and viral shedding in the upper and lower respiratory tract) many models failed to replicate the development of severe clinical symptoms 9.

Human pre-clinical COVID-19 lung models have also been developed in an attempt to overcome the shortcomings present with the aforementioned pre-clinical animal models¹⁰⁻¹². Notably, none of the pre-clinical human models recapitulate the heterogeneous epithelial cellularity of both the proximal and distal airways (i.e., airway epithelia, basal cells, secretory club cells and alveolar pneumocytes). Also noteworthy is that models derived from induced pluripotent stem cells (iPSCs) lack propagability and/or cannot be reproducibly generated for biobanking, nor can they be scaled up in cost-effective ways for use in drug screens.

Other than the aforementioned models, there are several additional approaches commonly used to model COVID-19 infection. For example, 3D organoids from bronchospheres and tracheospheres, which were previously established¹³⁻¹⁵, are now being used in apical-out cultures for infection with SARS-COV-2¹⁶. 3D airway models have also been generated from iPSCs or tissue-resident stem cells¹⁹⁻²⁴, while others have generated hiPSC-derived alveolar type-II (AT2) cells from iPSCs using closely overlapping protocols of sequential differentiation, including definitive endoderm, anterior foregut endoderm, and distal alveolar expression²⁵⁻³⁰. The air-liquid interphase (ALI model) is also commonly used for drug screening. The ALI model uses pseudo-stratified primary bronchial or small airway epithelial cells to recreate the multilayered mucociliary epithelium^{17,18}. Notably, long term in vitro culture conditions for pseudo-stratified airway epithelium organoids, derived from healthy and diseased adult humans suitable to assess virus infectivity³¹⁻³³, have been developed, but these airway organoids expressed virtually no lung mesenchyme or alveolar signature. It remains unclear if any of these models accurately recapitulate the immunopathologic phenotype seen in the lungs of COVID-19 infected patients.

### SUMMARY OF THE INVENTION

In one aspect, a composition and method for modeling a biological process in a human lung is provided. The method includes providing a culture composition and infecting the culture composition with a respiratory pathogen. The culture composition includes human lung proximal airway epithelial cells and distal airway epithelial cells.

In another aspect, a method of identifying a therapeutic agent effective for treating a respiratory disease is provided. This method includes providing a culture composition, infecting the culture composition with a respiratory pathogen, administering the therapeutic agent to the culture composition, and assessing whether the therapeutic agent is effective in treating or preventing the respiratory disease caused by the infection in the lung model. The therapeutic agent may be administered to the culture composition before or after the culture composition is infected with the respiratory pathogen. The culture composition includes human lung proximal airway epithelial cells and distal airway epithelial cells.

In yet another aspect, a human lung organoid model is provided. The human lung organoid model includes a culture composition. The culture composition includes human lung proximal airway epithelial cells and distal airway epithelial cells.

### BRIEF DESCRIPTION OF THE DRAWINGS

FIG. 1A depicts a whisker plot of relative levels of ACE2 expression in various cell types in a normal human lung.

FIG. 1B depicts immunofluorescence analysis of FFPE sections of a normal human lung and a lung of deceased COVID-19 patient.

FIG. 1C depicts a schematic of the key steps for generating an adult stem cell-derived lung organoid model.

FIG. 1D depicts a schematic of a transcriptome-based approach for cross-validation of in vitro lung models.

FIG. 1E depicts a whisker plot display of relative levels of TMPRSS2 expression in various cell types in a normal human lung.

FIG. 1F depicts stained FFPE sections of a lung of a deceased COVID-19 patient.

FIG. 1G depicts immunofluorescence analysis of FFPE sections of a normal human lung and a lung of deceased COVID-19 patient.

FIG. 1H depicts confocal immunofluorescence analysis of FFPE sections of a lung of deceased COVID-19 patient.

FIG. 1I depicts stained FFPE sections of a lung of a deceased COVID-19 patient.

FIG. 2A depicts a schematic the lung cell types and corresponding cell markers used to confirm the presence of each cell type.

FIG. 2B depicts bar graphs displaying the relative abundance of cell type markers in adult lung organoids, as compared to the airway and/or alveolar control cells where appropriate.

FIG. 2C depicts a schematic of the preparation of stained cell blocks using HistoGel.

FIG. 2D depicts immunofluorescence microscopy of the stained cell blocks.

FIG. 2E depicts immunofluorescence microscopy of the stained cell blocks.

FIG. 2F depicts a schematic of key demographics of patients serving as lung tissue donors.

FIG. 2G depicts bright-field microscopy of organoids grown in different conditions.

FIG. 2H depicts bright-field microscopy of organoids, imaged serially over a period of seven days.

FIG. 2I depicts bright-field microscopy of organoids at different passages.

FIG. 2J depicts stained cuts of HistoGel-embedded organoids.

FIG. 2K depicts bar graphs displaying the relative abundance of cell type markers in adult lung organoids, as compared to the airway and/or alveolar control cells where appropriate.

FIG. 2L depicts immunofluorescence microscopy of stained adult lung organoids.

FIG. 2M depicts a schematic of comparing cell type specific transcripts in adult lung organoids as compared to adult lung tissue.

FIG. 2N depicts bar graphs displaying the relative abundance of cell type markers in adult lung organoids, as compared to adult lung tissue.

FIG. 2O depicts a schematic of the comparison of cell type specific transcripts of early and late passages of adult lung organoids.

FIG. 2P depicts bar graphs displaying the relative abundance of cell type markers in adult lung organoids, comparing early and late passages.

FIG. 2Q depicts cell type marker gating and the representative marker values.

FIG. 3A depicts a schematic of the steps of adult lung organoid isolation and expansion from two types of monolayers.

FIG. 3B depicts bar graphs displaying the relative proportion of various lung cell types present at each step and for each sample type.

FIG. 3C depicts immunofluorescence microscopy of adult lung organoid monolayers grown in transwells.

FIG. 3D depicts immunofluorescence microscopy of adult lung organoid monolayers grown as an Air-Liquid Interface model.

FIG. 3E depicts microscopy of stained adult lung organoid monolayers that were challenged with COVID-19.

FIG. 3F depicts a whisker plot of infectivity of adult lung models infected with COVID-19.

FIG. 3G depicts a whisker of infectivity of adult lung models pretreated and subsequently infected with COVID-19.

FIG. 3H depicts a representation of adult lung organoid monolayers grown in transwells.

FIG. 3I depicts a representation of adult lung organoid monolayers grown as an Air-Liquid Interface.

FIG. 3J depicts immunofluorescence microscopy of adult lung organoid monolayers grown in transwells.

FIG. 3K depicts immunofluorescence microscopy of adult lung organoid monolayers grown as an Air-Liquid Interface.

FIG. 3L depicts adult lung organoid monolayers grown in transwells challenged with LPS and left untreated, as compared to monolayers challenged with and treated for LPS.

FIG. 3M depicts visualized small airway epithelial cell monolayers.

FIG. 3N depicts visualized bronchial epithelial cell monolayers.

FIG. 3O depicts the growth and visualization of human induced pluripotent stem cell-derived monolayers.

FIG. 3P depicts visualized monolayers challenged with COVID-19.

FIG. 3Q depicts a comparison of expression of viral envelope gene in monolayers infected with and not infected with COVID-19.

FIG. 3R depicts a Whisker plot of the change in viral envelope gene expression over time.

FIG. 4A depicts a schematic of the process of genetic analysis and comparison of healthy and COVID-19 infected human lung organoids.

FIG. 4B depicts a heat map representation of gene expression patterns.

FIG. 4C depicts a bar plot representing the ability of the differentially expressed genes to classify human lung organoids infected with COVID-19, as compared to published in vitro COVID-19 models.

FIG. 4D depicts the relative accuracy of the human lung organoid models infected with COVID-19 as models for COVID-19 infection.

FIG. 4E depicts a reactome pathway analysis of differentially expressed genes in lung autopsies for normal lungs, as compared to the lungs of COVID-19 patients.

FIG. 5A depicts a schematic of the process of genetic analysis and comparison of healthy and COVID-19 infected human lung organoids.

FIG. 5B depicts a heat map representation of gene expression patterns.

FIG. 5C depicts a comparison of the ability of the differentially expressed genes to classify human lung organoids infected with COVID-19, as compared to published in vitro COVID-19 models.

FIG. 5D depicts the relative accuracy of the human lung organoid models infected with COVID-19 as models for COVID-19 infection.

FIG. 5E depicts a reactome pathway analysis of differentially expressed genes in lung autopsies for normal lungs, as compared to the lungs of COVID-19 patients.

FIG. 5F depicts a comparison of the adult lung organoid model of COVID-19 as compared to existing COVID-19 models.

FIG. 6A depicts a schematic of the immune signatures identified via gene induction.

FIG. 6B depicts publicly available RNA sequencing data from commonly used lung models.

FIG. 6C depicts RNA sequencing data from human lung organoid models.

FIG. 6D depicts a summary of the findings disclosed herein, and the relationship of these findings to clinical representation of COVID-19 and modeling the same.

### DETAILED DESCRIPTION

Various further aspects and embodiments of the disclosure are provided by the following description. Before further describing various embodiments of the presently disclosed inventive concepts in more detail by way of exemplary description, examples, and results, it is to be understood that the presently disclosed inventive concepts are not limited in application to the details of methods and compositions as set forth in the following description. The presently disclosed inventive concepts are capable of other embodiments or of being practiced or carried out in various ways. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that the presently disclosed inventive concepts may be practiced without these specific details. In other instances, features which are well known to persons of ordinary skill in the art have not been described in detail to avoid unnecessary complication of the description. All of the compositions and methods of production and application and use thereof disclosed herein can be made and executed without undue experimentation in light of the present disclosure.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, 2^{nd} ed. (Sambrook et al., 1989): Oligonucleotide Synthesis (M. J. Gait, ed., 1984): Animal Cell Culture (R. I. Freshney, ed., 1987): Methods in Enzymology (Academic Press, Inc.): Current Protocols in Molecular Biology (F. M. Ausubel et al., eds., 1987, and periodic updates): PCR: The Polymerase Chain Reaction (Mullis et al., eds., 1994): Remington, The Science and Practice of Pharmacy, 20^{th} ed., (Lippincott, Williams & Wilkins 2003), and Remington, The Science and Practice of Pharmacy, 22^{th} ed., (Pharmaceutical Press and Philadelphia College of Pharmacy at University of the Sciences 2012).

As used herein, the terms “comprises,” “comprising,” “includes,” “including.” “has,” “having,” “contains”, “containing,” “characterized by,” or any other variation thereof, are intended to encompass a non-exclusive inclusion, subject to any limitation explicitly indicated otherwise, of the recited components. For example, a cell, a pharmaceutical composition, and/or a method that “comprises” a list of elements (e.g., components, features, or steps) is not necessarily limited to only those elements (or components or steps), but may include other elements (or components or steps) not expressly listed or inherent to the cell, pharmaceutical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause: other elements (or components) are not excluded from the claim as a whole.

As used herein, the transitional phrases “consists essentially of” and “consisting essentially of” are used to define a fusion protein, pharmaceutical composition, and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

It is understood that aspects and embodiments of the invention described herein include “consisting” and/or “consisting essentially of” aspects and embodiments.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Values or ranges may be also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In embodiments, “about” can be used to mean, for example, within 10% of the recited value, within 5% of the recited value, or within 2% of the recited value.

The present invention provides compositions and methods, useful as research tools for example, for identifying biological processes in and agents effective for treating respiratory diseases and infections due to pathogens, comprising a culture composition comprising human lung proximal airway epithelial cells and distal airway epithelial cells.

The invention provides methods of modeling a biological process in a human lung, the method comprising providing a culture composition comprising human lung proximal airway epithelial cells and distal airway epithelial cells; and infecting the culture composition with a respiratory pathogen.

In embodiments, the invention further provides determining a resulting biological process or a treatment effective response by a candidate therapeutic agent administered to the composition.

In embodiments, the invention provides methods of identifying a therapeutic agent effective for treating a respiratory disease, the method comprising administering a candidate therapeutic agent to the cell culture composition before or after the infecting step; and assessing whether the therapeutic agent is effective in treating or preventing the respiratory disease caused by the infection in the lung model.

In embodiments, the culture composition is derived from a monolayer of stem cell-derived adult lung organoids (ALOs). In embodiments, the culture composition comprises proximal airway epithelial cells that are proximal ciliated cells. In embodiments, the distal airway epithelial cells are distal alveolar cells. In embodiments, at least a portion of the distal alveolar cells are differentiated from alveolar type-II (AT2) pneumocytes to alveolar type-I (AT1) pneumocytes.

In embodiments, the respiratory pathogen is SARS-COV-2, such that infecting the culture composition creates a lung model of COVID-19 disease. In embodiments, the proximal airway epithelial cells permit viral infection. In embodiments, the distal alveolar cell differentiation permits viral propagation.

In embodiments, the method further comprises before the infecting step, analyzing the lung model for presence of viral entry markers angiotensin-converting enzyme-II (ACE2) and Transmembrane Serine Protease 2 (TMPRSS2).

In embodiments, the culture is grown in a conditioned media from L-WRN cells which express Wnt3, R-spondin and Noggin.

In embodiments, the invention provides a culture composition of a human lung organoid model derived from a monolayer of stem cell-derived adult lung organoids (ALOs) comprising primary airway cells and hiPSC-derived AT2 pneumocytes differentiated to AT1 pneumocytes; infecting the composition; administering a candidate agent to the composition; and determining a resulting biological process or a treatment effective response by the agent occurring in the composition.

In embodiments, the biological process is bacterial or viral infectivity, replication or host response. In embodiments, the infection is caused by administering a virus or bacteria. In embodiments, the virus is SARS-COV-2 to create an in vitro lung model of COVID-19.

In further embodiments, the compositions and methods are scalable, propagable, and personalized. In embodiments, the composition and method comprise or consist essentially of the components described herein.

In additional embodiments, the airway (proximal) cells permit sustained viral infection, and distal alveolar differentiation (AT2→AT1) permits a host immune response model of respiratory disease. In embodiments, the ALO monolayer is mixed with proximodistal airway components.

In one aspect, a method for modeling a biological process in a human lung is provided. The method includes providing a culture composition and infecting the culture composition with a respiratory pathogen. The culture composition includes human lung proximal airway epithelial cells and distal airway epithelial cells.

In another aspect, a method of identifying a therapeutic agent effective for treating a respiratory disease is provided. This method includes providing a culture composition, infecting the culture composition with a respiratory pathogen, administering the therapeutic agent to the culture composition, and determining whether the therapeutic agent is effective in treating or preventing the respiratory disease caused by the infection in the lung model. The therapeutic agent may be administered to the culture composition before or after the culture composition is infected with the respiratory pathogen. The culture composition includes human lung proximal airway epithelial cells and distal airway epithelial cells.

In yet another aspect, a human lung organoid model is provided. The human lung organoid model includes a culture composition. The culture composition includes human lung proximal airway epithelial cells and distal airway epithelial cells.

The term “model” or “modelling” refers to a non-naturally occurring research tool or procedure for observing effects analogous to a biological cell, organ or process such that positive correlations may be made therebetween. In embodiments of the invention, a human lung organoid model is provided.

The human lung organoid model includes a culture composition. The culture composition includes human lung proximal airway epithelial cells and distal airway epithelial cells. Tissues, cells, and their progeny of a biological entity obtained in vivo or cultured in vitro are also encompassed.

In embodiments, the human lung proximal airway epithelial cells and distal airway epithelial cells are provided in a non-naturally occurring concentration in the culture composition, also referred to as an enriched cell population. As used herein, a composition containing an enriched cell population means that at least 10%, 20%, 30%, 50%, 60%, 70%, 80%, 90%, 95%, 98%, 99%, of the cells in the composition are of the identified type.

Generally, techniques for differentiating an induced pluripotent cell involve modulation of specific cellular pathways, either directly or indirectly, using polynucleotide-, polypeptide- and/or small molecule-based approaches. The developmental potency of a cell may be modulated, for example, by contacting a cell with one or more modulators. “Contacting”, as used herein, can involve culturing cells in the presence of one or more factors (such as, for example, small molecules, proteins, peptides, etc.). In some embodiments, a cell is contacted with one or more agents to induce cell differentiation. Such contact, may occur for example, by introducing the one or more agents to the cell during in vitro culture. Thus, contact may occur by introducing the one or more agents to the cell in a nutrient cell culture medium. The cell may be maintained in the culture medium comprising one or more agents for a period sufficient for the cell to achieve the differentiation phenotype that is desired.

Differentiation of stem cells requires a change in the culture system, such as changing the stimuli agents in the culture medium or the physical state of the cells. A conventional strategy utilizes the formation of embryoid bodies (EBs) as a common and critical intermediate to initiate the lineage-specific differentiation. EBs are three-dimensional clusters that have been shown to mimic embryo development as they give rise to numerous lineages within their three-dimensional area. Through the differentiation process simple EBs (for example, aggregated pluripotent stem cells elicited to differentiate) continue maturation and develop into a cystic EB at which time, they are further processed to continue differentiation. EB formation is initiated by bringing pluripotent stem cells into close proximity with one another in three-dimensional multilayered clusters of cells. Typically, this is achieved by one of several methods including allowing pluripotent cells to sediment in liquid droplets, sedimenting cells into “U” bottomed well-plates or by mechanical agitation. To promote EB development, the pluripotent stem cell aggregates require further differentiation cues, as aggregates maintained in pluripotent culture maintenance medium do not form proper EBs. This may be followed by additional stimulation differentiating the iPSCs to hematopoietic cells and then to convert the hematopoietic progenitor cells into natural killer (NK).

As used herein, “differentiate” or “differentiated” are used to refer to the process and conditions by which immature (unspecialized) cells acquire characteristics becoming mature (specialized) cells thereby acquiring particular form and function. Stem cells (unspecialized) are often exposed to varying conditions (e.g., growth factors and morphogenic factors) to induce specified lineage commitment, or differentiation, of said stem cells. The process by which an unspecialized (“uncommitted”) or less specialized cell acquires the features of a specialized cell such as, for example, a blood cell or a muscle cell. A differentiated or differentiation-induced cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type.

Differentiation marker gene(s) may be monitored to gauge a cells state of differentiation. As used herein, the term “differentiation marker gene,” or “differentiation gene,” refers to genes whose expression are indicative of cell differentiation occurring within a cell, such as a pluripotent cell.

“Culture” or “cell culture” refers to the maintenance, growth and/or differentiation of cells in an in vitro environment. “Cell culture media,” “culture media” (singular “medium” in each case), “supplement” and “media supplement” refer to nutritive compositions that cultivate cell cultures.

“Cultivate,” or “maintain,” refers to the sustaining, propagating (growing) and/or differentiating of cells outside of tissue or the body, for example in a sterile plastic (or coated plastic) cell culture dish or flask. “Cultivation,” or “maintaining.” may utilize a culture medium as a source of nutrients, hormones and/or other factors helpful to propagate and/or sustain the cells.

In some embodiments, one or more of the media of the culture platform is a feeder-free environment, and optionally is substantially free of cytokines and/or growth factors. In some embodiments, the cell culture media contains supplements such as serums, extracts, growth factors, hormones, cytokines and the like. Generally, the culture platform comprises one or more of stage specific feeder-free, serum-free media, each of which further comprises one or more of the followings: nutrients/extracts, growth factors, hormones, cytokines and medium additives. Suitable nutrients/extracts may include, for example, DMEM/F-12 (Dulbecco's Modified Eagle Medium/Nutrient Mixture F-12), which is a widely used basal medium for supporting the growth of many different mammalian cells; KOSR (knockout serum replacement); L-glut; NEAA (Non-Essential Amino Acids). Other medium additives may include, but not limited to, MTG, ITS, (ME, anti-oxidants (for example, ascorbic acid). In some embodiments, a culture medium of the present invention comprises one or more of the following cytokines or growth factors: epidermal growth factor (EGF), acidic fibroblast growth factor (aFGF), basic fibroblast growth factor (bFGF), leukemia inhibitory factor (LIF), hepatocyte growth factor (HGF), insulin-like growth factor 1 (IGF-1), insulin-like growth factor 2 (IGF-2), keratinocyte growth factor (KGF), nerve growth factor (NGF), platelet-derived growth factor (PDGF), transforming growth factor beta (TGF-β), bone morphogenetic protein (BMP4), vascular endothelial cell growth factor (VEGF) transferrin, various interleukins (such as IL-1 through IL-18), various colony-stimulating factors (such as granulocyte/macrophage colony-stimulating factor (GM-CSF)), various interferons (such as IFN-γ) and other cytokines having effects upon stem cells such as stem cell factor (SCF) and erythropoietin (EPO). These cytokines may be obtained commercially, for example from R&D Systems (Minneapolis, Minn.), and may be either natural or recombinant. In some other embodiments, the culture medium of the present invention comprises one or more of bone morphogenetic protein (BMP4), insulin-like growth factor-1 (IGF-1), basic fibroblast growth factor (bFGF), vascular endothelial growth factor (VEGF), hematopoietic growth factor (for example, SCF, GMCSF, GCSF, EPO, IL3, TPO, EPO), Fms-Related Tyrosine Kinase 3 Ligand (Flt3L); and one or more cytokines from Leukemia inhibitory factor (LIF), IL3, IL6, IL7, IL11, IL15. In some embodiments, the growth factors/mitogens and cytokines are stage and/or cell type specific in concentrations that are determined empirically or as guided by the established cytokine art.

The term “human lung proximal airway epithelial cells” includes proximal ciliated cells that line the tracheobronchial airway passages in nature. The proximal airway epithelial cells permit viral infection.

In embodiments, the human lung organoid model is a culture composition of a derived from a monolayer of stem cell-derived adult lung organoids (ALOs) comprising primary airway cells and hiPSC-derived AT2 pneumocytes differentiated to AT1 pneumocytes. In embodiments, the model and methods provide analyzing the lung model for presence of viral entry markers angiotensin-converting enzyme-II (ACE2) and Transmembrane Serine Protease 2 (TMPRSS2).

The term “distal airway epithelial cells” includes distal alveolar cells. The distal alveolar cells permit viral propagation. In embodiments, at least a portion of the distal AT2 cells are differentiated to AT1 pneumocytes.

The term “respiratory pathogen” includes viral, bacterial, and fungal infectious agents. Viral infectious agents are the most common cause of respiratory infection, and include rhinoviruses, respiratory syncytial virus, influenza virus, parainfluenza virus, human metapneumovirus, measles, mumps, adenovirus, and coronaviruses. In embodiments, the viral infectious agent is SARS-COV-2.

In another aspect, a method of identifying a therapeutic agent effective for treating a respiratory disease is provided. This method includes providing a culture composition, infecting the culture composition with a respiratory pathogen, administering the therapeutic agent to the culture composition, and determining whether the therapeutic agent is effective in treating or preventing the respiratory disease caused by the infection in the lung model. The therapeutic agent may be administered to the culture composition before or after the culture composition is infected with the respiratory pathogen.

As used herein, “patient” or “subject” means a human or animal subject to be treated.

As used herein the term “pharmaceutical composition” refers to pharmaceutically acceptable compositions, wherein the composition comprises a pharmaceutically active agent, and in some embodiments further comprises a pharmaceutically acceptable carrier. In some embodiments, the pharmaceutical composition may be a combination of pharmaceutically active agents and carriers.

As used herein the term “pharmaceutically acceptable” means approved by a regulatory agency of the Federal or a state government or listed in the U.S. Pharmacopoeia, other generally recognized pharmacopoeia in addition to other formulations that are safe for use in animals, and more particularly in humans and/or non-human mammals.

As used herein, “therapeutically effective amount” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with diseases or conditions. For example, an effective amount in reference to diseases is that amount which is sufficient to block or prevent onset: or if disease pathology has begun, to palliate, ameliorate, stabilize, reverse or slow progression of the disease, or otherwise reduce pathological consequences of the disease. In any case, an effective amount may be given in single or divided doses.

As used herein, the terms “treat,” “treatment,” or “treating” embraces at least an amelioration of the symptoms associated with diseases in the patient, where amelioration is used in a broad sense to refer to at least a reduction in the magnitude of a parameter, e.g. a symptom associated with the disease or condition being treated. As such, “treatment” also includes situations where the disease, disorder, or pathological condition, or at least symptoms associated therewith, are completely inhibited (e.g. prevented from happening) or stopped (e.g. terminated) such that the patient no longer suffers from the condition, or at least the symptoms that characterize the condition.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. In certain embodiments, subjects with familial history of a disease are potential candidates for preventive regimens. In certain embodiments, subjects who have a history of recurring symptoms are also potential candidates for prevention. In this regard, the term “prevention” may be interchangeably used with the term “prophylactic treatment.”

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with one or more other agent(s), which provides a prophylactic benefit in the prevention of the disease. The term “prophylactically effective amount” can encompass an amount that improves overall prophylaxis or enhances the prophylactic efficacy of another prophylactic agent.

### EXAMPLES

It will be understood from the foregoing description that various modifications and changes may be made in the various embodiments of the present disclosure without departing from their true spirit. The description provided herein is intended for purposes of illustration only and is not intended to be construed in a limiting sense. Thus, while the presently disclosed inventive concepts have been described herein in connection with certain embodiments so that aspects thereof may be more fully understood and appreciated, it is not intended that the presently disclosed inventive concepts be limited to these particular embodiments. On the contrary, it is intended that all alternatives, modifications and equivalents are included within the scope of the presently disclosed inventive concepts as defined herein. Thus the examples described above, which include particular embodiments, will serve to illustrate the practice of the presently disclosed inventive concepts, it being understood that the particulars shown are by way of example and for purposes of illustrative discussion of particular embodiments of the presently disclosed inventive concepts only and are presented in the cause of providing what is believed to be a useful and readily understood description of procedures as well as of the principles and conceptual aspects of the inventive concepts. Changes may be made in the construction and formulation of the various components and compositions described herein, the methods described herein or in the steps or the sequence of steps of the methods described herein without departing from the spirit and scope of the presently disclosed inventive concepts.

### Example 1: A Rationalized Approach for Creating and Validating Acute Lung Injury in COVID-19

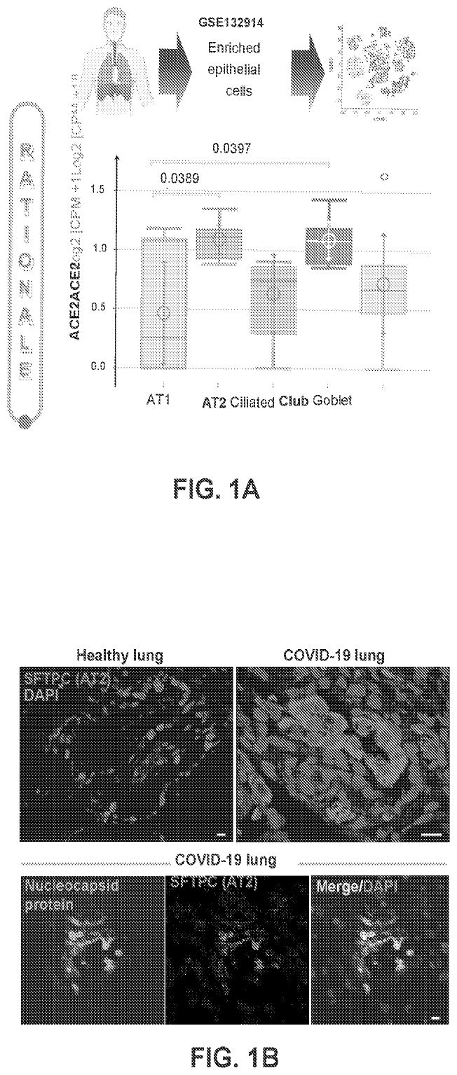

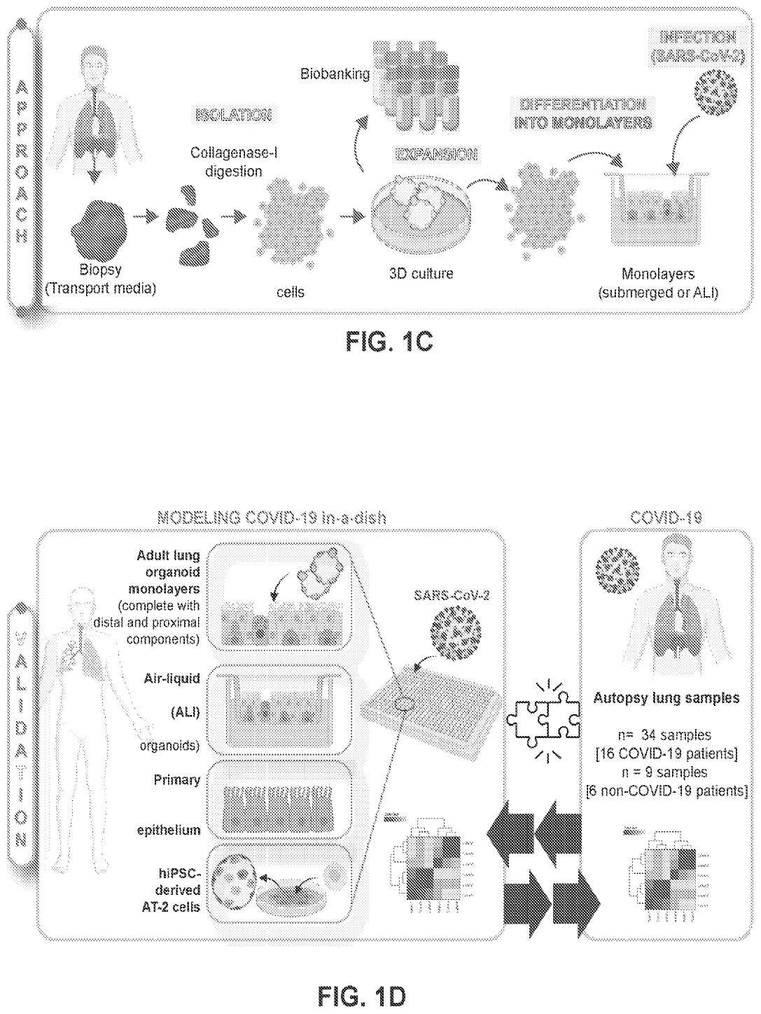

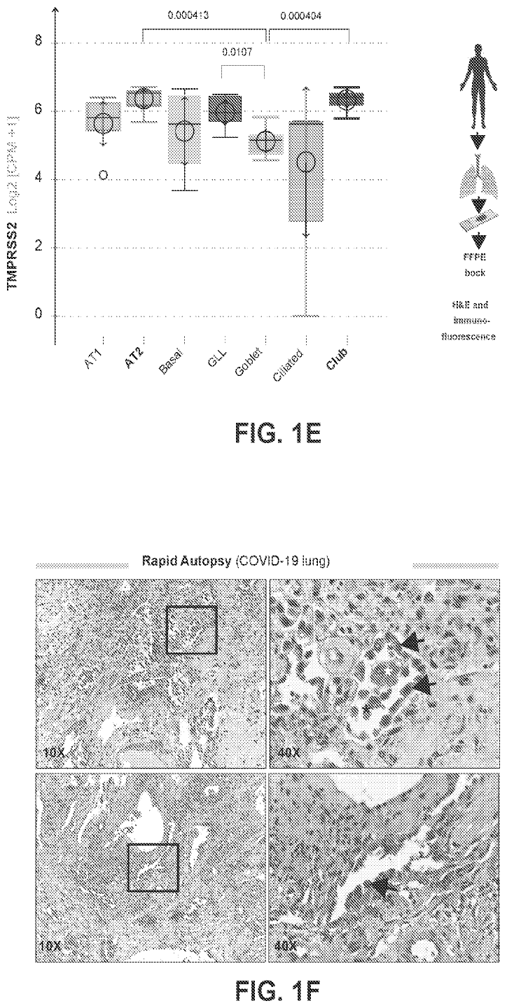

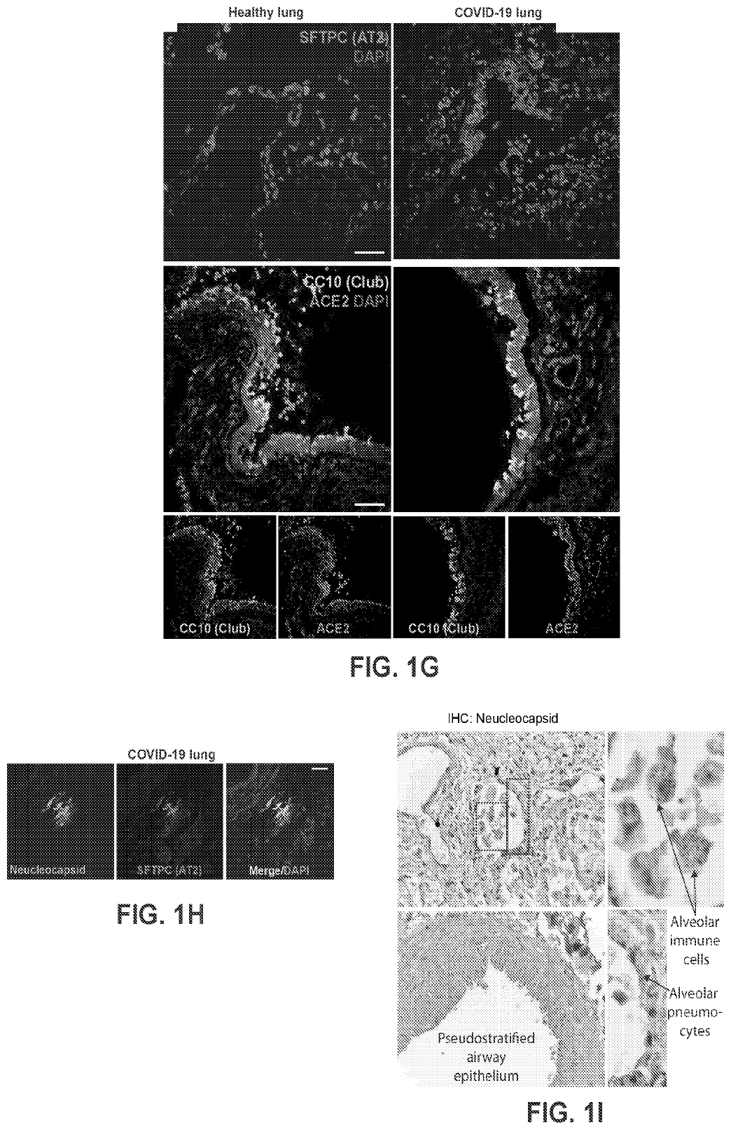

As shown in the schematic included in FIG. 1A, human lung single-cell sequencing dataset GSE132914 was analyzed for levels of expression of angiotensin-converting enzyme-II (ACE2) and Transmembrane Serine Protease 2 (TMPRSS2) to determine which cell types in the lungs may be most readily infected by COVID-19. ACE2 and TMPRSS2 are two receptors that have been shown to be the primary sites of entry for the SARS-COV-2³⁴. The dataset was queried with widely accepted markers of all the major cell types.

To determine which cell types most prominently expressed ACE2 and TMPRSS2, the various cell types were annotated within the GSE132914 dataset and the p-values were analyzed by one-way Anova and Tukey post hoc test. As shown in the plots of FIGS. 1A and 1E, alveolar epithelial type 2 (AT2), ciliated, and club cells emerged as the cell types with the highest expression of ACE2 and TMPRSS2 in the normal human lung. These observations are consistent with published studies demonstrating that ACE2 is indeed expressed highest in AT2 and ciliated cells^{11,35,36}.

Turning to FIG. 1B, stained formalin-fixed paraffin-embedded (FFPE) sections of the human lung from normal and deceased COVID-19 patients are shown. The FFPE sections were stained for surfactant protein-C(SFTPC), alone or in combination with nucleocapsid protein. The stained FFPE sections were and analyzed by confocal immunofluorescence to produce the representative images.

In some deceased COVID-19 patients, it was observed that gas-exchanging flatted AT1 pneumocytes are virtually replaced by cuboidal cells. This was observed in H&E stained FFPE sections of the human lung from deceased COVID-19 patients, as shown in FIG. 1F. However, the immunofluorescent staining of FIG. 1B confirmed that the cells were in fact AT2-like cells.

This finding is highlighted further in FIGS. 1G-1H, where FFPE sections of the human lung from normal and deceased COVID-19 patients were stained for AT2 and club cell marks, such as ACE2 or viral nucleocapsid protein. Confocal immunofluorescence confirmed that club cells also express ACE2, underscoring the importance of preserving these cells in lung models of COVID-19 infection. The presence of SARS-COV-2 in alveolar pneumocytes was also confirmed, as determined by the colocalization of viral nucleocapsid protein with SFTPC. Immunohistochemistry studies, as shown in FIG. 1I, further showed the presence of SARS-COV-2 virus in alveolar pneumocytes and in alveolar immune cells.

These findings are all consistent with the general consensus in the field that alveolar pneumocytes support the interaction between the epithelial cells and inflammatory cells recruited to the lung. Although the mechanisms of interaction remain unclear, it is believed that this interaction contributes to the development of acute lung injury and acute respiratory distress syndrome (ARDS), and the severe hypoxemic respiratory failure during COVID-19^{37,38}.

Because prior work has demonstrated that SARS-COV-2 infectivity in patient-derived airway cells is highest in the proximal airway epithelium compared to the distal alveolar pneumocytes (AT1 and AT2)³⁷, and yet, it is the AT2 pneumocytes that harbor the virus, and the AT1 pneumocytes that are ultimately destroyed during diffuse alveolar damage, we hypothesized that both proximal airway and distal (alveolar pneumocyte) components might play distinct roles in the respiratory system to mount the so-called viral infectivity and host immune response phases of the clinical symptoms observed in COVID-19³⁹.

Turning to FIG. 1C, a schematic demonstrating the key steps for generating an adult stem cell-derived lung organoid model is depicted. Notably, the adult lung organoid model disclosed herein includes both proximal and distal airway components, which are used to model COVID-19 infection.

Because existing lung models do not provide proximodistal cellular representation, such models may not accurately recapitulate the clinical phases of COVID-19. Thus, a primary objective in developing the lung models disclosed herein was to develop a lung model having both proximal and distal airway epithelia, derived from adult stem cells isolated from deep lung biopsies (i.e., biopsies sufficient to reach the bronchial tree). Lung organoids were generated according to the workflow depicted in FIG. 1C, following methodologies from a modified published porotocol^{31,33}. Organoids grown in 3D cultures were subsequently dissociated into single cells to create 2D monolayers, which were either submerged in media or used in an air-liquid interface (ALI) model. The 2D monolayers were later infected with SARS-COV-2 infection, and an RNA sequence analysis was performed.

Referring now to FIG. 1D, a transcriptome-based approach for cross-validating in vitro lung models of SARS-COV-2 infection as compared to COVID-19 infection in humans is shown. The objective of this approach is to identify matches in gene expression signatures. Primary airway epithelial cells and hiPSC-derived alveolar type-II (AT2) pneumocytes were also used as additional models. Each transcriptomic dataset was subsequently used to cross-validate the ex-vivo lung models of SARS-COV-2 infection disclosed herein with the human COVID-19 autopsy lung specimens. This comparison objectively assed whether each model was capable of accurately recapitulating the gene expression signatures in the patient-derived lungs.

### Example 2: Creation of a Lung Organoid Model with Both Proximal and Distal Airway Epithelia

The adult stem cell-derived lung organoids, having both proximal and distal airway components, disclosed herein are propagatable models.

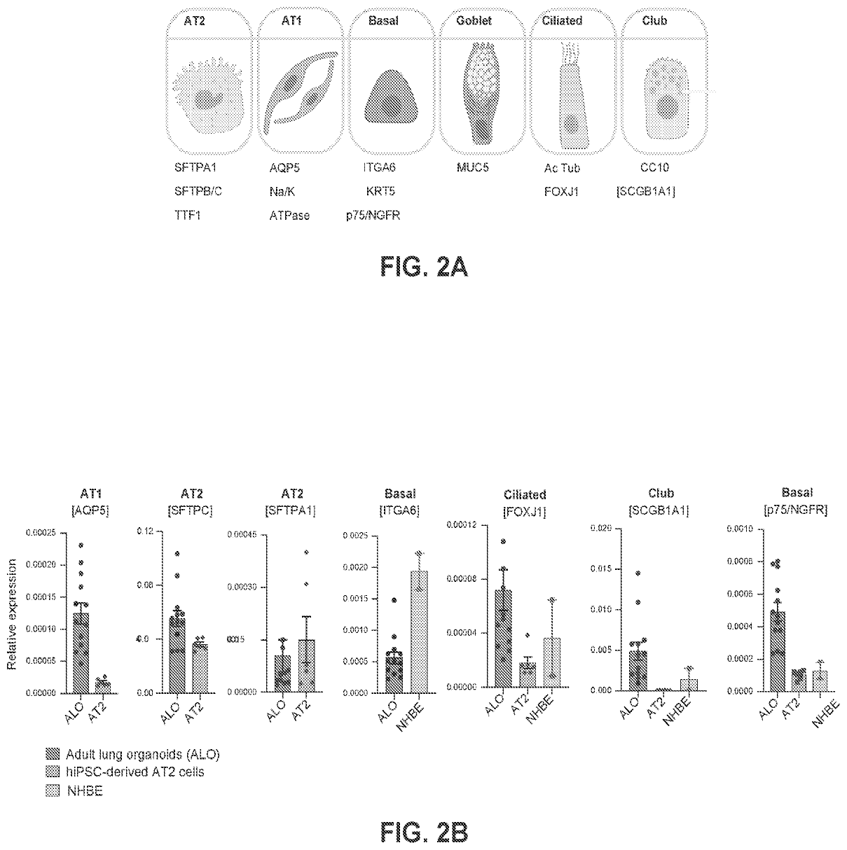

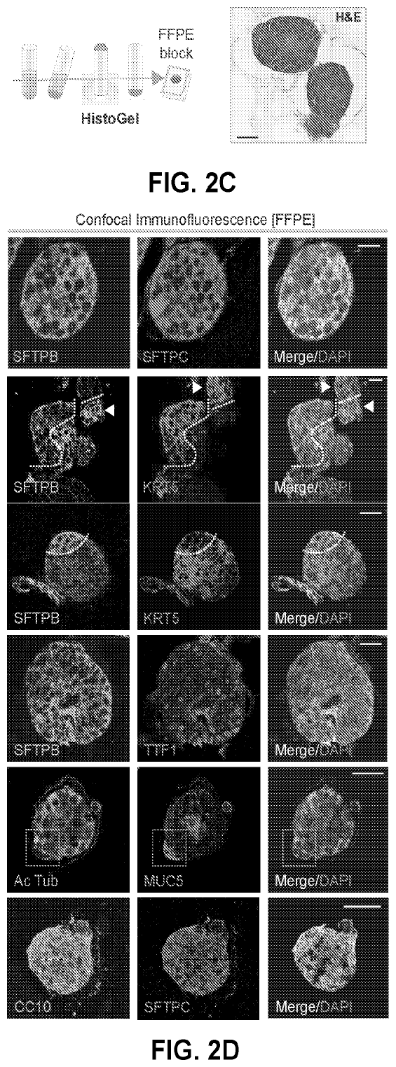

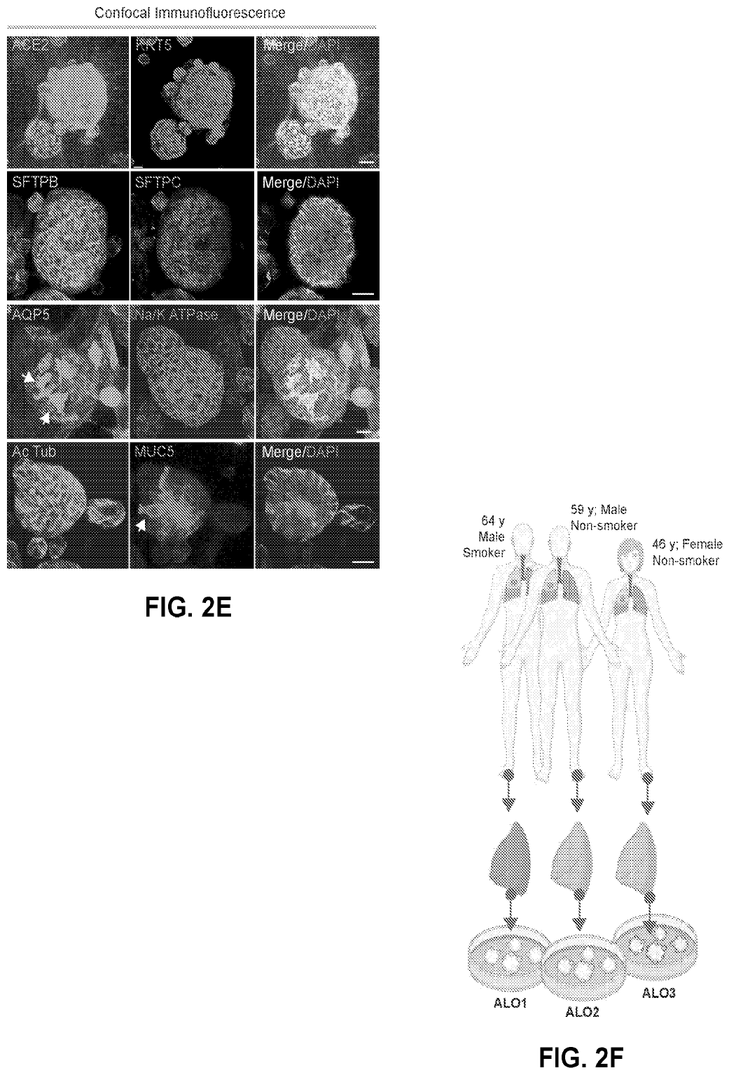

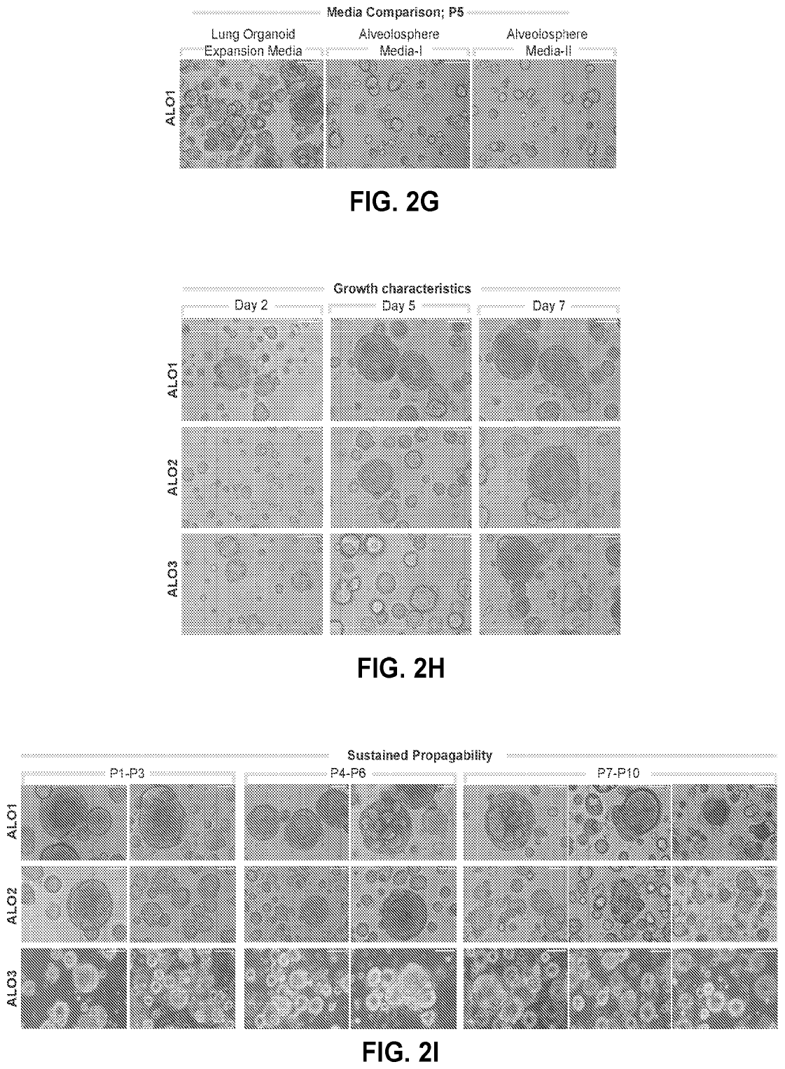

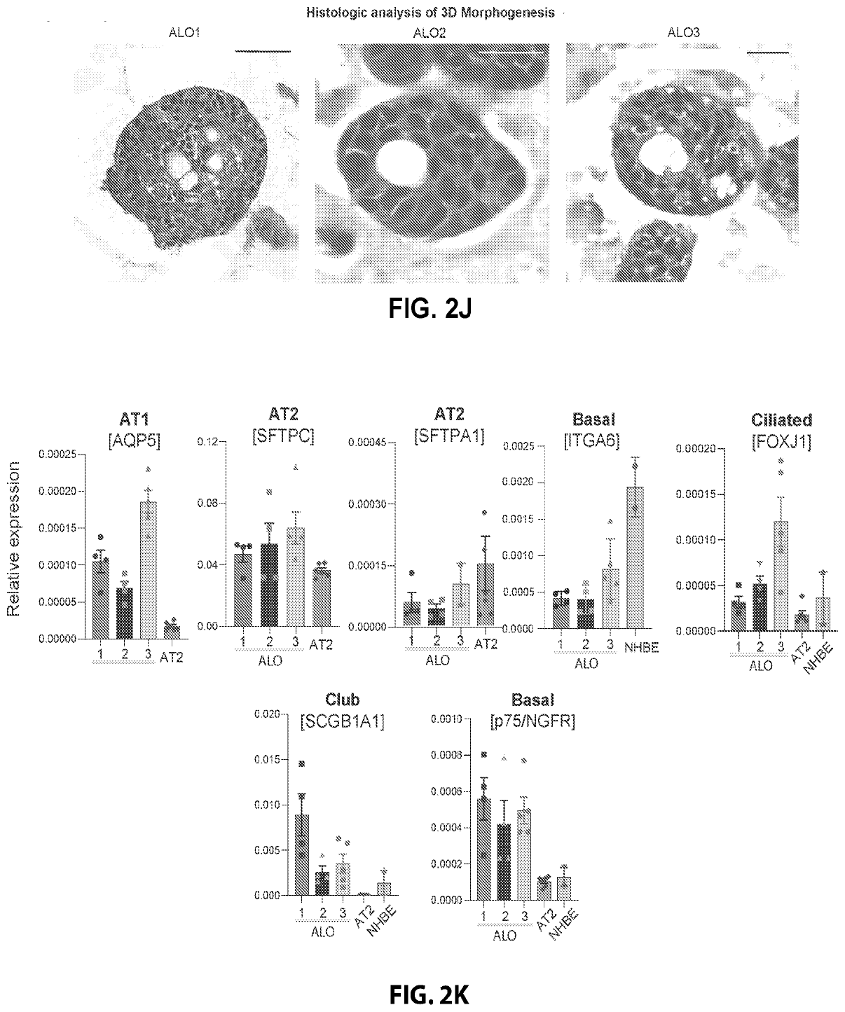

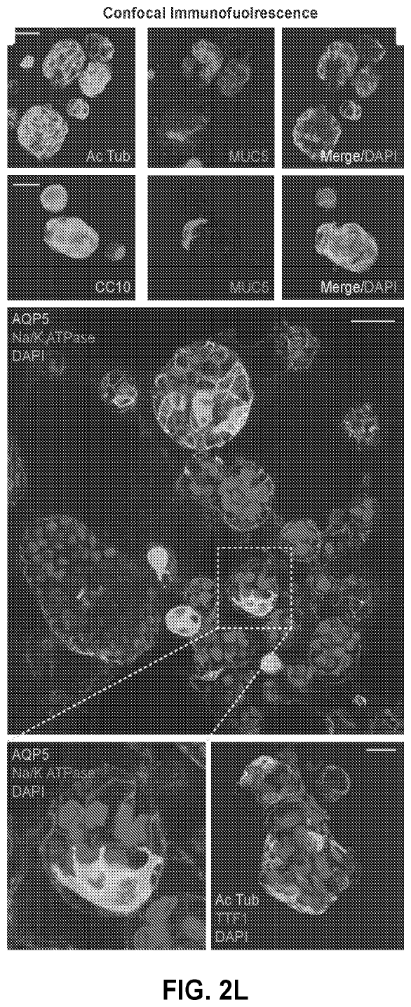

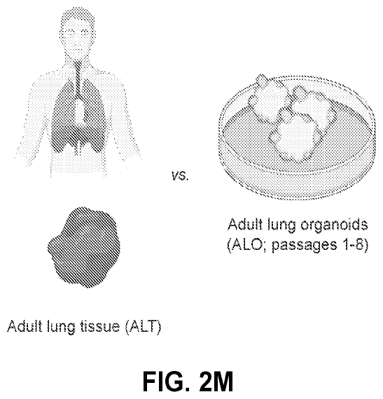

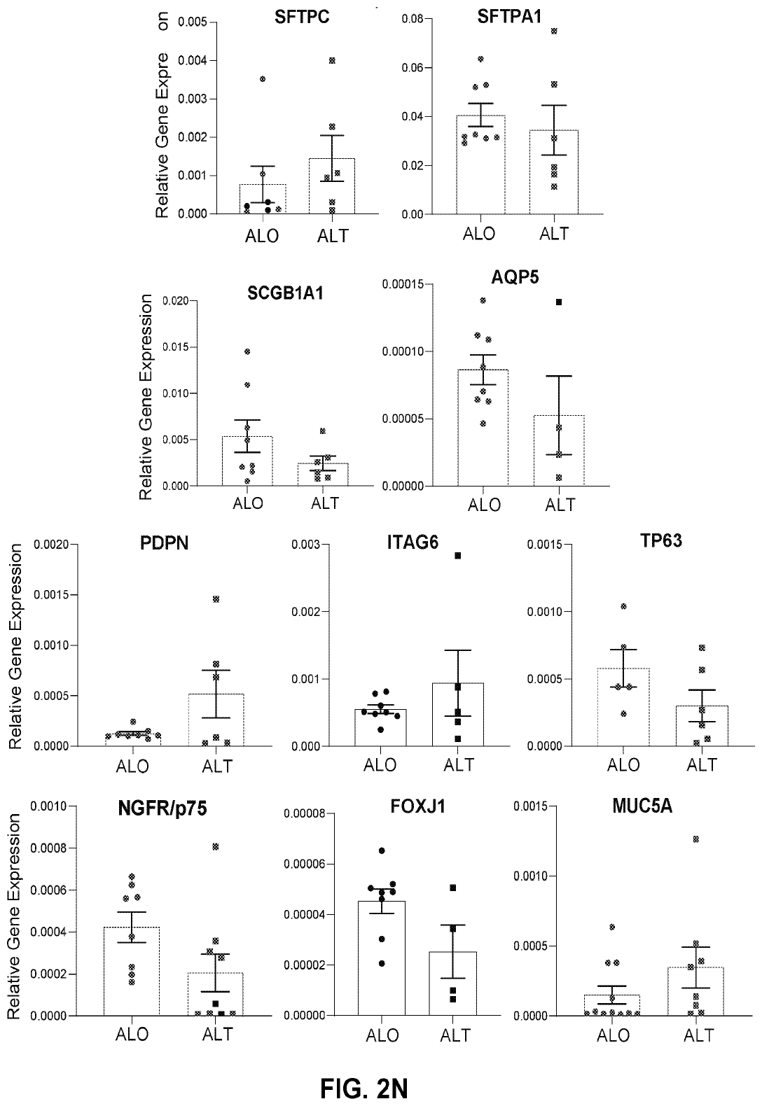

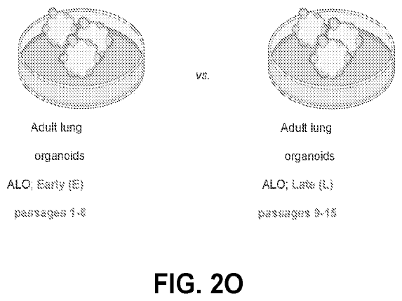

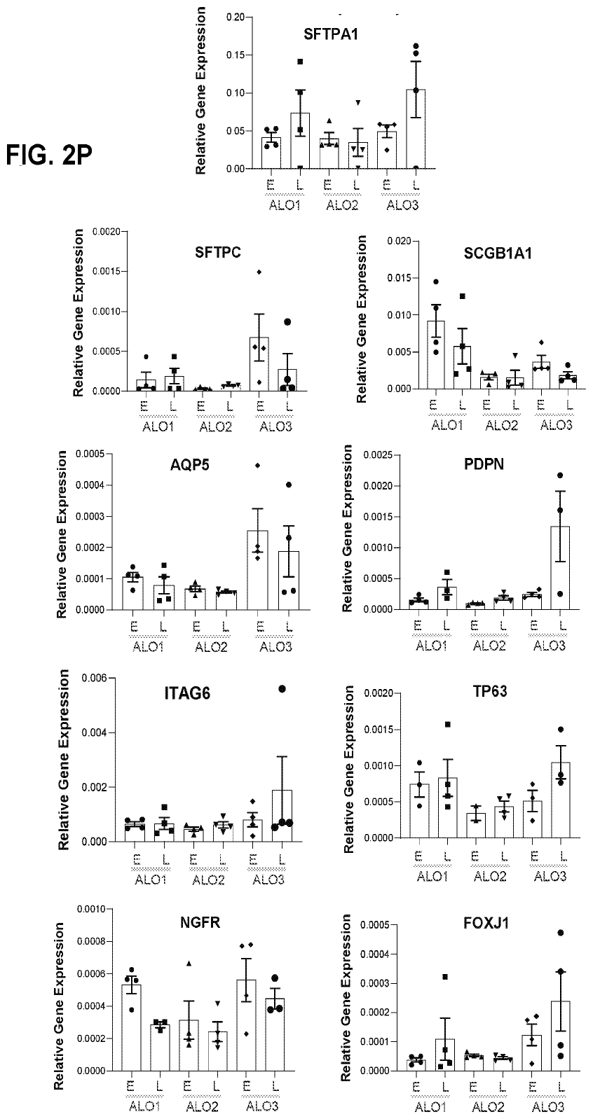

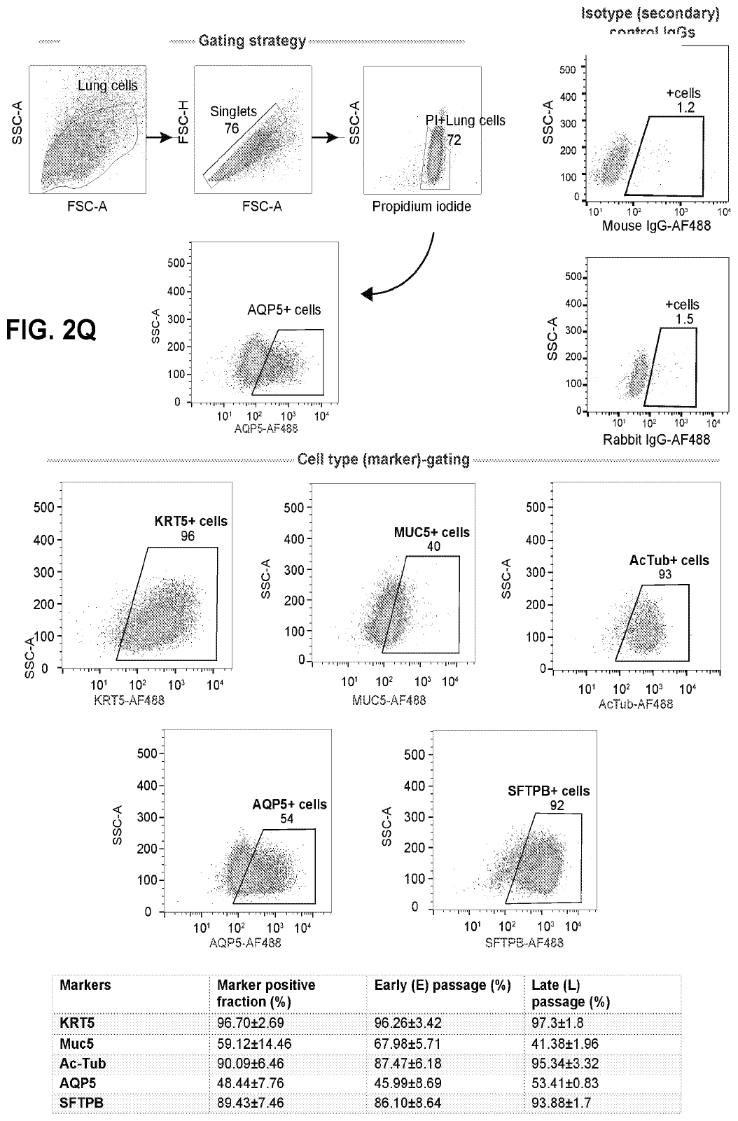

Three lung organoid lines were developed from deep lung biopsies obtained from the normal regions of lung lobes surgically resected from patients being treated for lung cancer. FIG. 2F depicts a schematic representation of the donor patient demographics. As shown, both genders, smokers, and non-smokers were represented.

Referring now to FIG. 2G, organoids were propagated in three different types of media and compared using bright-field microscopy. The three media were selected by the ability of the media to support adult-stem cell-derived mixed epithelial cellularity in other organs, like the gastrointestinal tract⁴⁰⁻⁴², or rationalized based on published growth conditions for proximal and distal airway components^{25,31,32}. A growth condition that included conditioned media from L-WRN cells which express Wnt3, R-spondin and Noggin, supplemented with recombinant growth factors, referred to herein as “lung organoid expansion media,” was superior as compared to alveolosphere media-I and II^{27,28} Notably, the lung organoid expansion media was able to consistently and reproducibly support the best morphology and growth characteristics across multiple attempts to isolate organoids from lung tissue samples.

As shown in FIG. 2H, three adult lung organoid lines (ALO1-3) were developed using the lung organoid expansion media. The adult lung organoid lines were monitored for their growth characteristics by bright-field microscopy over a serios of 10 days. The adult lung organoid lines of FIG. 2H were also cultured with similar phenotypes at different passages, up to P10, as shown in FIG. 2I. Referring to FIG. 2J, serial cuts of HistoGel-embedded organoids were analyzed by H&E staining. The H&E staining was effective to assess the 3D morphology of the lung organoids.

Turning to FIG. 2A, the relevant lung epithelial cell types and corresponding cell type markers are shown. The relevant cell types for the purpose of the models disclosed herein include AT2, AT1, basal, goblet, ciliated, and club cells. The corresponding cell type markers were used for qPCR and immunofluorescence to confirm the presence of all cell types in both the 3D lung organoids and 2D monolayers.

The cell type markers were analyzed by qRT-PCR, which confirmed that all six relevant cell types were present in the lung organoids. The bar graphs of FIG. 2B (and FIG. 2K) depict the (normalized) relative abundance of the cell type markers in the lung organoids. The abundance of the cell type markers was also compared with the airway (NHBE) and/or alveolar (AT2) control cells as necessary. One-way annova was used to analyze the respective p-values of these comparisons.

All three lung organoid lines had a comparable level of AT2 cell surfactant markers, compared against hiPSC-derived AT2 cells as positive control, and a significant amount of AT1, as determined using the marker AQP5. The lung organoids also contained basal cells, as determined by the marker ITGA6 and p75/NGFR, ciliated cells, as determined by the marker FOXJ1, club cells, as determined by the marker SCGB1A1, and stem cells, as determined by marker TP63. As expected, the primary human bronchial epithelial cells (NHBE) had significantly higher expression of basal cell markers than the ALO lines, but lacked stemness and club cells.

Turning to FIGS. 2C, H&E stained lung organoid cell blocks were prepared using HistoGel. Referring to FIG. 2D, each cell block slide was stained for the aforementioned markers and visualized by confocal immunofluorescence microscopy. Specifically, 3D lung organoids grown in 8-well chamber slides were also fixed, immunostained, and visualized by confocal microscopy.

The presence of all six cell types was also confirmed by assessing protein expression of the cell types within organoids grown in 3D cultures. As discussed with respect to FIGS. 2C-2D, protein expression was confirmed using slices cut from FFPE cell blocks of HistoGel-embedded lung organoid lines. Alternatively, as shown in FIG. 2E, lung organoids were grown in 8-well chamber slides were fixed in matrigel, stained, and assessed by confocal microscopy. This staining not only confirmed the presence of all cell types in each lung organoid line but also demonstrated the presence of more than one cell type (i.e., mixed cellularity) of proximal (basal-KRT5) and distal (AT1/AT2 markers) within the same organoid structure.

For example, AT2 and basal cells marked by SFTPB and KRT5, respectively, were found in the same 3D structure. Similarly, ciliated cells and goblet cells stained by Ac-Tub and Muc5, respectively, were also found to coexist within the same structure. Notably, Intriguingly, 3D structures having cells that co-stained for CC10 and SFTPC likely represent a unique population of multipotent stem cells termed bronchioalveolar stem cells (BASCs), which have been found to be located at the bronchioalveolar-duct junctions (BADJs)^{43,44}. Besides the organoids with heterogeneous makeup, each lung organoid line also showed homotypic organoid structures that were relatively enriched in one cell type. Regardless of homotypic or heterotypic cellular organization into 3D structures, the presence of mixed cellularity was documented in all three lung organoid lines, which is also shown in FIG. 2L.

Adult stem cell-derived lung organoids generally recapitulate cell type specific gene expression patterns observed in the adult lung tissue from which they originate. A schematic of this analysis, that is comparing the cell type transcripts in lung organoids versus lung tissue, is shown in FIG. 2M. The bar graphs of FIG. 2N display the (normalized) relative abundance of the cell type markers in adult lung organoids from early passage, as compared to the adult lung tissue from which the organoids were derived. One-way Anova was used to analyze the respective p-values. No statistically significant differences were noted.

Comparison of cell type transcripts in early and late passages of the lung organoid lines is also relevant, and depicted in FIG. 2O. The bar graphs of FIG. 2P display the (normalized) relative abundance of the early passages, as compared to the late passages. One-way Anova was used to analyze the respective p-values. No statistically significant differences were noted.

Adult stem cell-derived lung organoids having both a proximal and distal airway epithelial population generally maintain diversity from early to late passages. Referring now to FIG. 2Q, lung monolayers were dissociated into single cells and analyzed using flow cytometry. The flow cytometry findings are consistent with the findings of others using multi-channel FACS¹¹⁴, showing that although many markers are highly expressed in a certain cell type, the markers are shared at lower levels among other cell types.

Using qRT-PCR of various cell-type markers as a measure, it was confirmed that the adult lung organoid models recapitulated the cell type composition in the adult lung tissues from which they were derived. This composition was also retained in later passages without significant notable changes in any particular cell type. The mixed proximal and distal cellular composition of the lung organoid models, and the degree of stability during in vitro culture, was also confirmed by flow cytometry.

### Example 3: Organoid Cellularity Resembles Tissue Sources in 3D Cultures but Differentiates in 2D Cultures

To model respiratory infections such as COVID-19, it is necessary for pathogens to be able to access the apical surface. It is possible to microinject into the lumens of 3D organoids, as done previously with pathogens in the case of gut organoids⁴⁵⁻⁴⁸ or FITC-dextran in the case of lung organoids⁴⁹, or to carry out infection in apical-out 3D lung organoids with basal cells¹². However, most researchers have gained apical access by dissociating 3D organoids into single cells and plating the cells as 2D-monolayers^{10,11,31,33,50-52}. As in any epithelium, the differentiation of airway epithelial cells relies upon dimensionality, orapicobasal polarity. This is because the loss of dimensionality can have a major impact on cellular proportions, impacting disease-modeling in unpredictable ways.

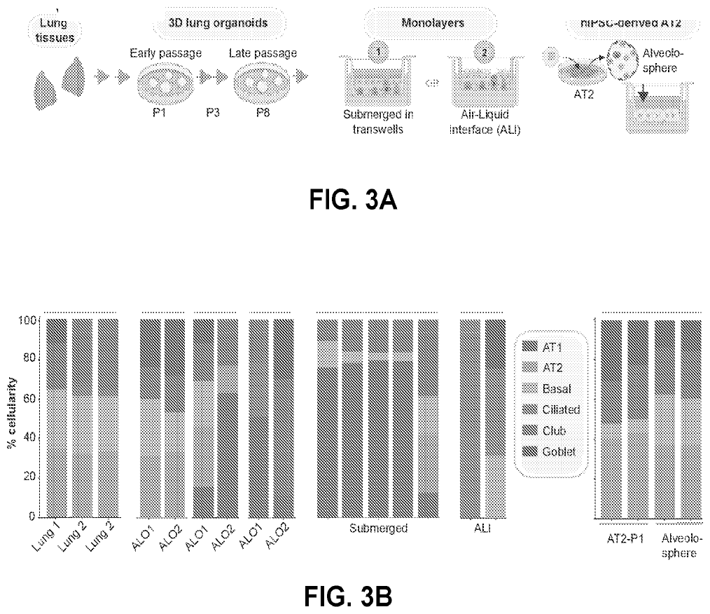

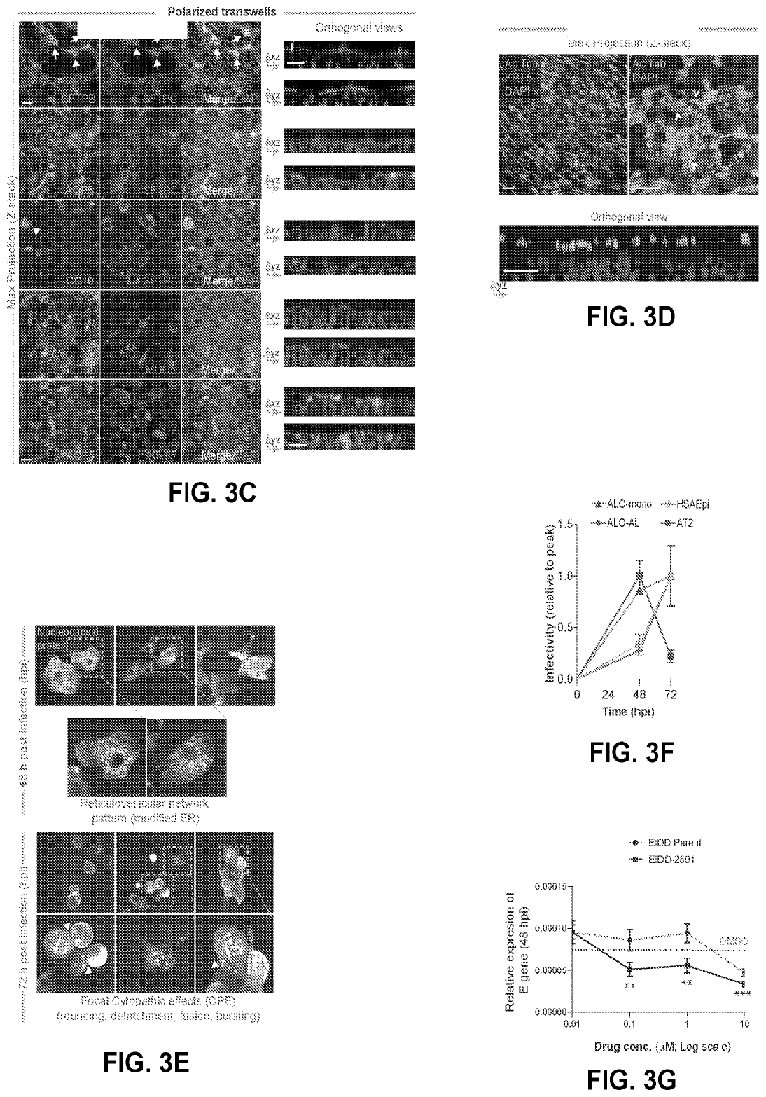

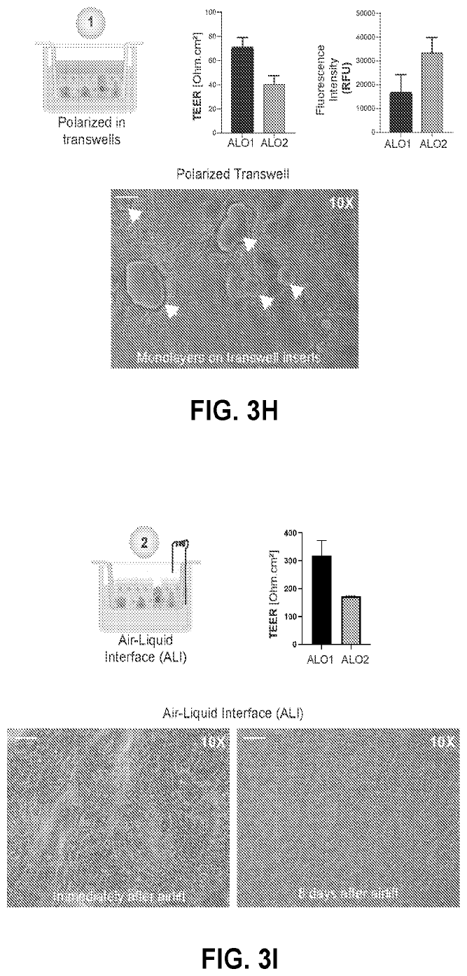

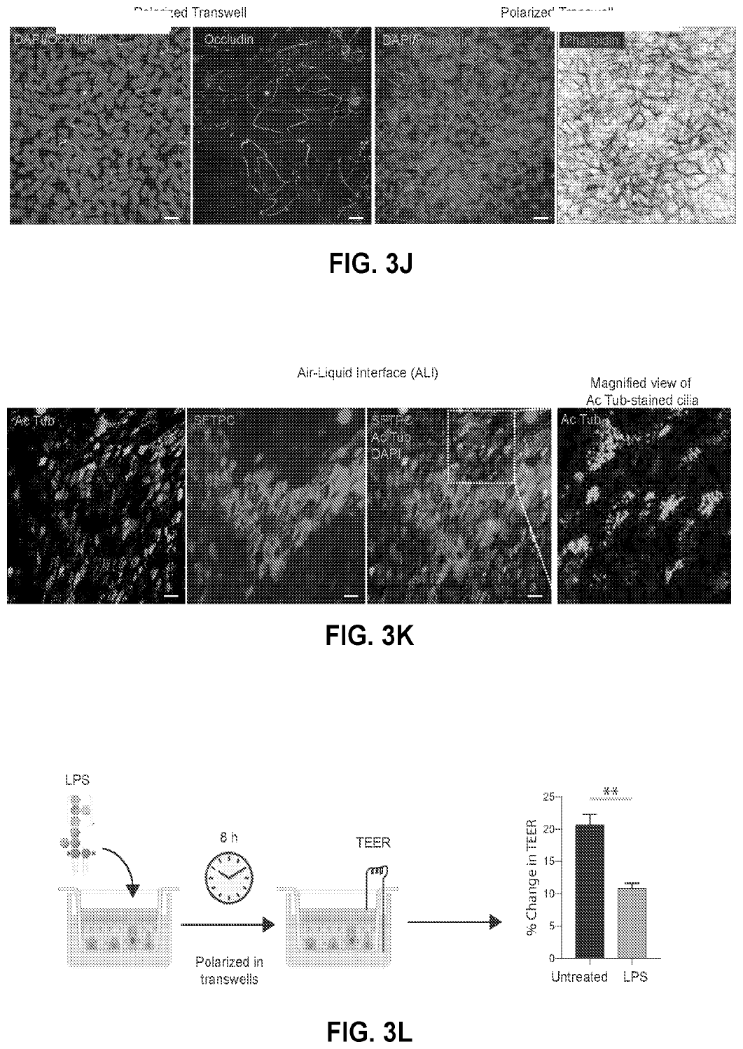

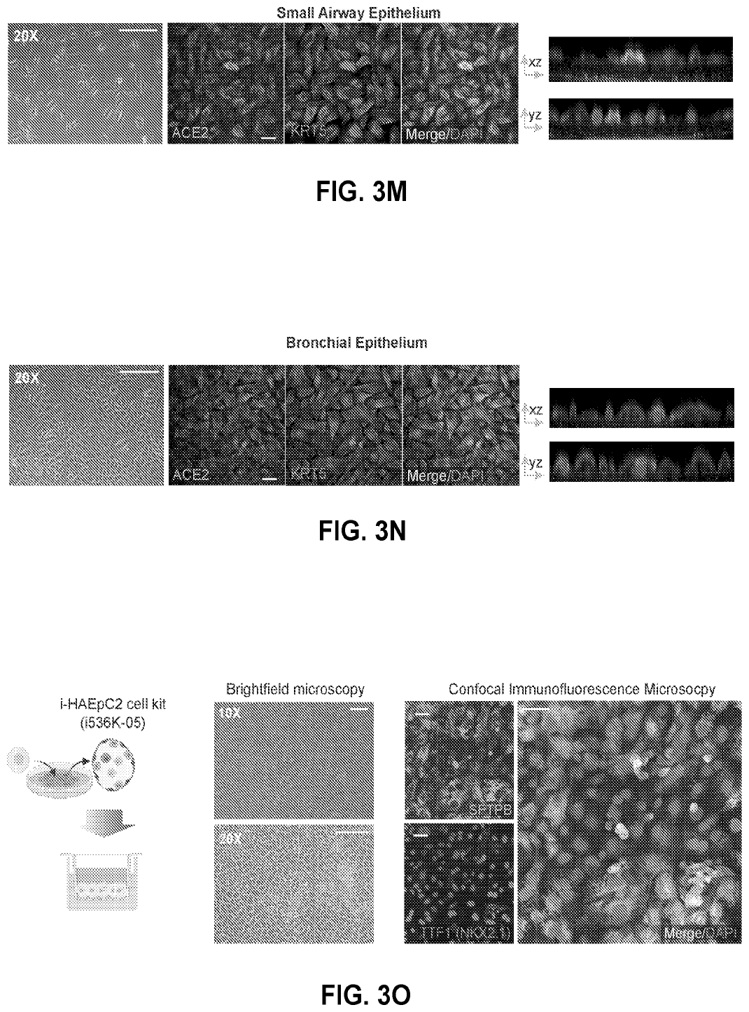

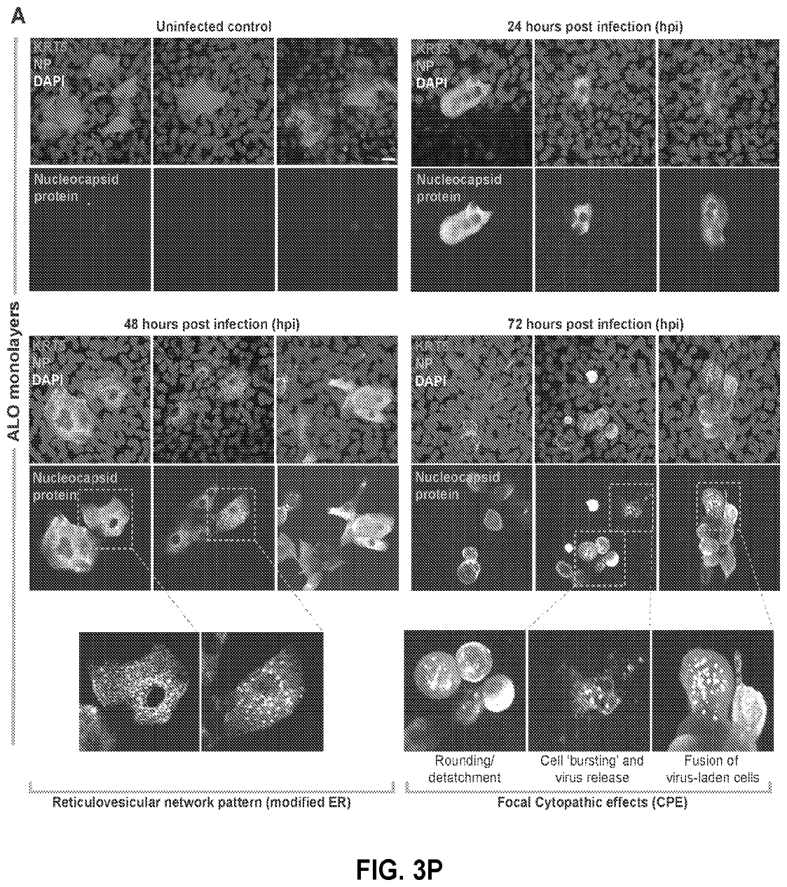

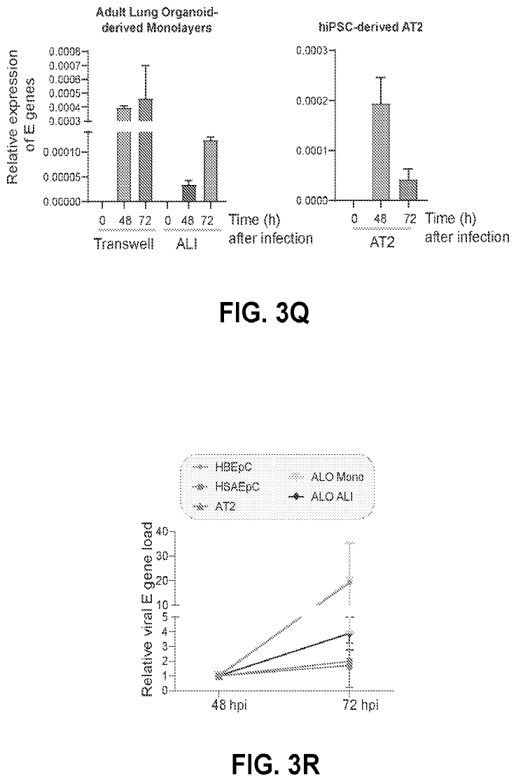

Referring to FIG. 3A, samples collected at various steps of lung organoid isolation and expansion in culture, and from the two types of monolayers prepared using the lung organoids, are shown. RNA sequencing was used to analyze these samples, and the datasets were compared for the respective percentages of cellular compositions, as shown in FIG. 3B.

Monolayers derived from adult lung organoids can form an epithelial barrier. Two commonly encountered methods of growth in 2D monolayers were tested. The first method involves polarizing monolayers on trans-well inserts but submerged in growth media, as shown in FIG. 3H. The second method involves growing monolayers at the air-liquid interface, known as an ALI model^{53,54}, for 21-days to differentiate into the mucociliary epithelium, as shown in FIG. 3I. The submerged 2D monolayers had several regions of organized vacuolated-appearing spots, presumably due to morphogenesis and cellular organization even in 2D. The epithelial barrier was leakier, as determined by relatively lower trans-epithelial electrical resistance and the flux of FITC-dextran from apical to basolateral chambers, and corroborated by morphological assessment by confocal immunofluorescence of localization of occludin, a bona-fide TJ marker. Occludin was used because it is a shared and constant marker throughout the airway that stabilizes claudins and regulates their turnover⁵⁵ and plays an important role in maintaining the integrity of the lung epithelial barrier⁵⁶.

As shown in FIGS. 3J-3K, submerged monolayers were fixed with methanol (FIG. 3J) or paraformaldehyde (FIG. 3K) prior to co-staining, and were visualized using immunofluorescence. The immunofluorescence showed that junction-localized occludin was patchy in the monolayer, despite the fact that the monolayer was otherwise intact, as determined by phalloidin staining.

The observation that 3D organoids, differentiated into monolayers in submerged cultures, where alveolar differentiation and cell-flattening happens dynamically as progenitor cells give rise to AT1/AT2 cells, are leaky is in keeping with prior work demonstrating that TJs are rapidly remodeled as alveolar cells mature^{57,58} By contrast, and as expected⁵⁹, the ALI-monolayers formed a more effective epithelial barrier, as determined by TEER, and appeared to be progressively hazier with time after air-lift, likely due to the accumulation of secreted mucin.

RNA sequence datasets were analyzed using the same set of cell markers described above. Consistent with the morphologic, gene expression, and FACS-based studies previously described, cell-type deconvolution of the transcriptomic dataset, using CIBERSORTx (cibersortx.stanford.edu/runcibersortx.php), confirmed that cellular composition in the human lung tissues was reflected in the 3D lung organoid models. The cell type composition was also relatively well-preserved over several passages. Both the tissue and organoid models showed a mixed population of simulated alveolar, basal, club, ciliated and goblet cells. When 3D organoids were dissociated and plated as 2D monolayers on transwells, the AT2 signatures were virtually abolished with a concomitant and prominent emergence of AT1 signatures, suggesting that growth in 2D-monolayers favor differentiation of AT2 cells into AT1 cells⁶⁰. A compensatory reduction in proportion was also observed for the club, goblet and ciliated cells.

The same organoids, when grown in long-term 2D culture conditions in the ALI model, showed a strikingly opposite pattern. Specifically, alveolar signatures were almost entirely replaced by a concomitant increase in ciliated and goblet cells. These findings are consistent with the well-established notion that ALI conditions favor growth as pseudo-stratified mucociliary epithelium^{53,54}.

Also depicted in FIGS. 3A-3B, hiPSC-derived AT2 cells and alveolospheres were plated as monolayers and analyzed by RNA sequencing. As an alternative model for use as monolayers for viral infection, hiPSC-derived AT2 cells and alveolospheres were developed using established protocols⁵¹. Because hiPSC-derived AT2 cells and alveolosphers were grown in the presence of CHIR99021, an aminopyrimidine derivate that is a selective and potent Wnt agonist 27.28.61 that likely inhibits the AT2 to AT1 differentiation, these monolayers were enriched in AT2 and devoid of AT1 cells.

Turning to FIGS. 3C-3D, submerged lung organoid monolayers in transwells (FIG. 3C) and monolayers grown as ALI models (FIG. 3D) were fixed and stained for cell type markers and visualized by confocal immunofluorescence microscopy. FIGS. 3C-3D represent the representative maximum projected Z-stack images and the corresponding orthogonal images.

The multicellularity of lung organoid monolayers was confirmed by immunofluorescence staining and confocal microscopy of the submerged and ALI monolayers. As expected, markers for the same cell type (i.e., SFTPB and SFTPC, both AT2 markers) colocalize, but markers for different cell types do not. Submerged monolayers showed the prominent presence of both AT1 (AQP5-positive) and AT2 cells. Compared to the submerged monolayers, the ALI model showed a significant increase in the ciliated epithelium. This increase was associated with a concomitant decrease in KRT5-stained basal cells. This loss of the basal cell marker KRT5 between submerged monolayers and the ALI model can be attributed to and the expected conversion of basal cells to other cell types (i.e., ciliated cells)^{62,63}. The presence of AT2 cells, scattered amidst the ciliated cells in these ALI monolayers, was confirmed by co-staining them for SFTPC and Ac-Tub (see FIG. 3K).

As shown in FIG. 3L, submerged monolayers were challenged with 500 ng/ml LPS to confirm that the epithelial barrier is responsible to infections. Compared to unchallenged controls, the integrity of the barrier was impaired by LPS, as indicated by a significant drop in the TEER, which is in keeping with the known disruptive role of LPS on the respiratory epithelium⁶⁴.

Taken together, the immunofluorescence images are in agreement with the RNA sequence dataset. That is, both demonstrate that the short-term submerged monolayer favors distal differentiation (AT2 to AT1), whereas the 21-day ALI model favors proximal mucociliary differentiation. Notably, these distinct differentiation phenotypes originated from the same 3D-organoids despite the seeding of cells in the same basic media composition (i.e., PneumaCult) prior to switching over to an ALI-maintenance media for the prolonged growth at Air-Liquid interface. The latter is a well-described methodology that promotes differentiation into ciliated and goblet cells⁵⁹.

### Example 4: Differentiated 2D-Monolayers Show that SARS-COV-2 Infectivity is Higher in Proximal than Distal Epithelia

As shown in FIG. 3E, monolayers of a lung organoid line were challenged with SARS-COV-2. The monolayers were challenged prior to fixation and staining for KRT5, SARS-COV2-viral nucleocapsid protein, and DAPI. The monolayers were subsequently visualized by confocal microscopy, which demonstrated the reticulovesicular network patterns and various cytopathic effects. Referring to FIG. 3F, monolayers of lung organoid hiPSC-derived AT2 cells, and other alternative models, were infected with SARS-COV-2 and analyzed for infectivity by qPCR, targeting amplification of viral envelope, E gene. The infected samples were compared with non-infected samples.

Because the lung organoids with complete proximodistal cellularity could be differentiated into either proximal-predominant monolayers in submerged short-term cultures or distal-predominant monolayers in long-term ALI cultures. This differentiation is effective to model the respiratory tract and assess the impact of the virus along the entire proximal-to-distal gradient.

To assess the viral impact, it is first necessary to inquire as to whether the lung organoid monolayers are permissive to SARS-COV-2 infection and replication. Confocal imaging of lung organoid monolayers infected with the anti-SARS-COV-2 nucleocapsid protein antibody showed that submerged lung organoid monolayers showed progressive changes during the 48 to 7 hour window after infection. By 48 hours after infection, formation of reticulovesicular patterns indicative of viral replication within modified host endoplasmic reticulum⁶⁵ were observed. And by 72 hours after infection, focal cytopathic effect (CPE)⁶⁶, such as cell-rounding, detachment, and bursting of virions, was observed. These observations are depicted in FIG. 3P.

It was also necessary to inquire as to how viral infectivity varies in the various lung models. Because multiple groups have shown the importance of the ciliated airway cells for infectivity, such as viral entry, replication and apical release^{37,67-69}, as positive controls, monolayers of human airway epithelia were infected. Representative immunofluorescence imaging of the infected monolayers and controls are depicted in FIGS. 3M-3N. AT2 cells, which express high levels of viral entry receptors ACE2 and TMPRSS2, been shown to be proficient in viral entry, but are least amenable to sustained viral release and infectivity^{37,69}. To this end, monolayers of hiPSC-derived homogeneous cultures of AT2 cells were as secondary controls, as shown in FIG. 3O.

Infection was carried out using the Washington strain of SARS-COV-2, USA-WA1/2020 (BEI Resources NR-5228170). As expected, both sets of 2D lung monolayers were readily infected with SARS-COV-2, as determined by the presence of E gene. Infectivity of the monolayers is shown in FIG. 3Q. However, the kinetics of viral amplification differed. When expressed as levels of E gene normalized to the peak values in each model, the kinetics of the ALI-monolayer model mirrored that of the primary airway epithelial monolayers. Both showed slow beginning in the first 48 hours, followed by an exponential increase in E gene levels from 48 to 72 hours. The submerged monolayer model showed sustained viral infection during the 48 to 72 hour window. In the case of AT2 cells, the 48 to 72 hour window was notably missing in monolayers of hiPSC-derived AT2 cells, also shown in FIG. 3Q.

When the kinetics of viral E gene expression was specifically analyzed during the late phase (i.e., the 48 to 72 hour window), it was observed that the proximal airway models (human Bronchial airway Epi (HBEpC)) were more permissive to viral replication than distal models (human Small Airway Epi (HSAEpC) and AT2), as shown in FIG. 3R. The lung organoid monolayers showed intermediate sustained infectivity, albeit with variability. All models showed extensive cell death and detachment by 96 hours and, hence, were not analyzed.

Using the E gene as a readout, it was determined whether lung organoid models could be used as platforms for pre-clinical drug screens. The efficacy of nucleoside analog N⁴-hydroxycytidine (NHC: EIDD-parent) and its derivative pro-drug. EIDD-2801, were tested. Both have been shown to inhibit viral replication, in vitro and in SARS-COV-2-challenged ferrets^{71,72}. Lung organoid monolayers plated in 384-wells were pre-treated for 4 hours with the compounds or DMSO, as a control, prior to infection. The monolayers were assessed at 48 hours post-infection for the abundance of E gene in the monolayers. As shown in FIG. 3G, both compounds effectively reduced the viral titer in a dose-dependent manner, and the pro-drug derivative showed a better efficacy.

Taken together, these findings show that sustained viral infectivity is best simulated in monolayers that resemble the proximal mucociliary epithelium, i.e., 2D-monolayers of lung organoids grown as ALI models and the primary airway epithelia. Because prior studies conducted in patient-derived airway cells³⁷ mirrors what we see in our monolayers, we conclude that proximal airway cells within the present mixed-cellular model appear to be sufficient to model viral infectivity in COVID-19. Findings also provide evidence that ALO monolayers may be adapted in miniaturized formats for use in 384-well plates for high-throughput (HTP) drug screens.

### Example 5: Differentiated 2D-Monolayers Show that Host Immune Response is Higher in Distal than Proximal Epithelia

It was determined whether the lung models disclosed herein accurately recapitulate the host immune response in COVID-19 infection. That is, the infected lung organoid monolayers, the airway epithelial (HSAEpC), and AT2 monolayers were analyzed by RNA sequencing. The RNA sequences were compared against the transcriptome profile of lungs from deceased COVID-19 patients. To complete this analysis, publicly available RNA sequence dataset (GSE151764 of lung autopsies from patients who were deceased due to COVID-19 or non-infectious were analyzed for differential expression of genes.

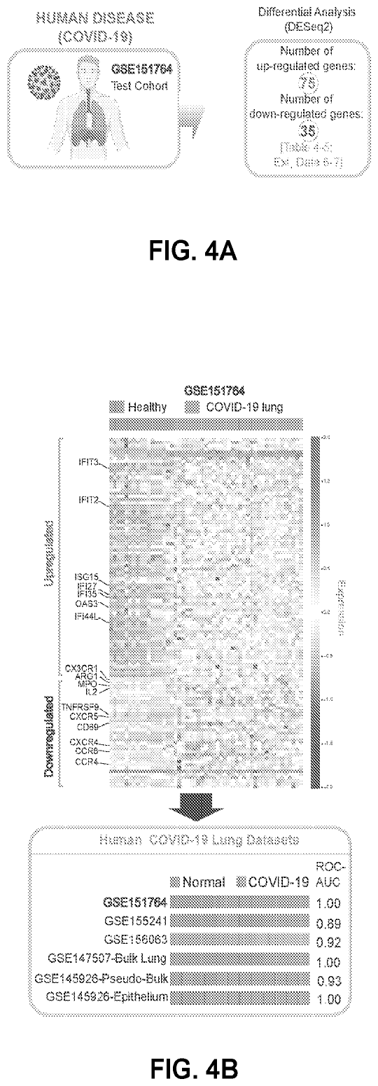

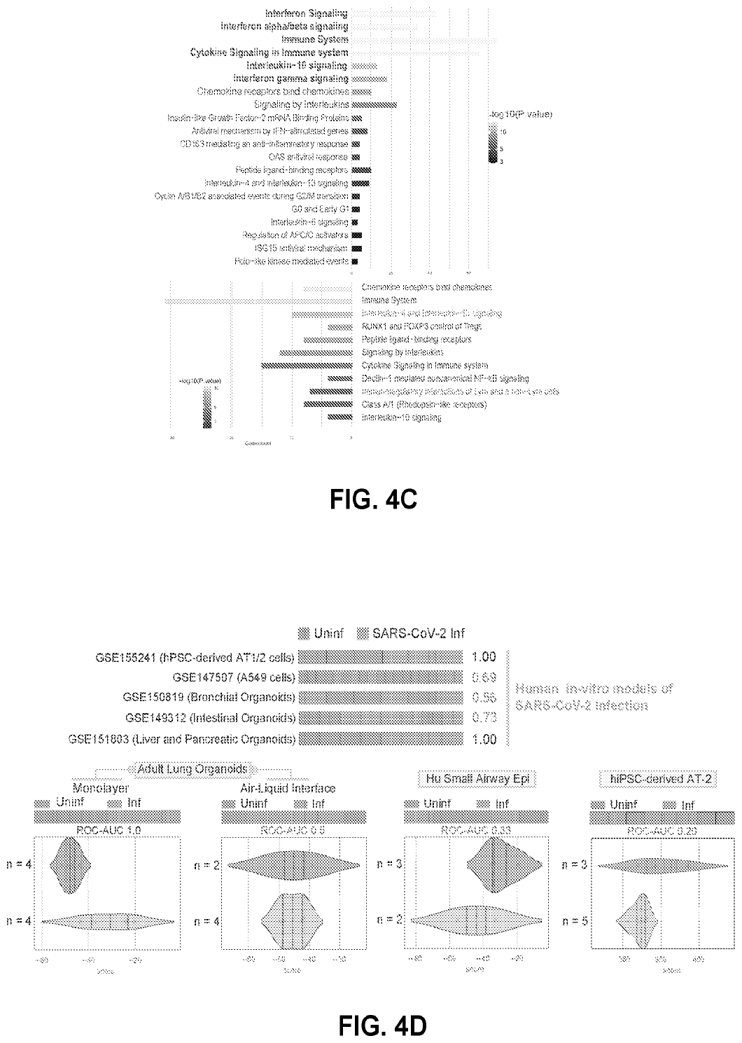

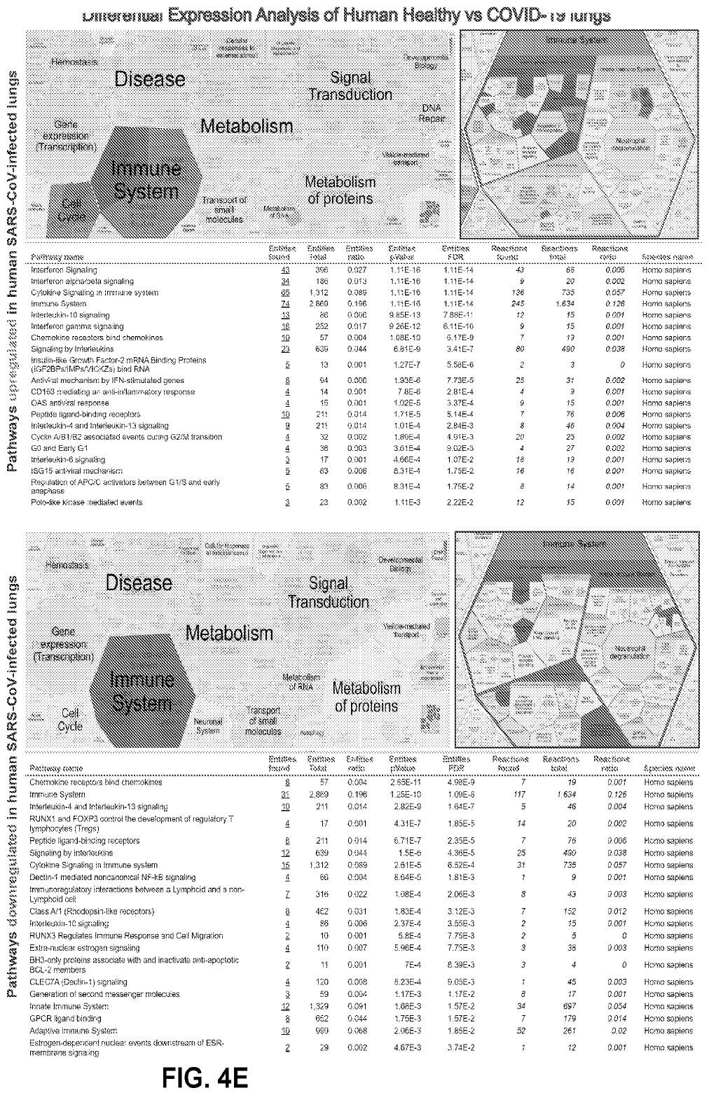

This analysis proceeded in two steps. First, the actual human disease-derived gene signature was assessed for its ability to distinguish infected from uninfected disease models. Second, the ALO model-derived gene signature was assessed for its ability to distinguish healthy from diseased patient samples. A publicly available dataset GSE15176473, comprised of lung transcriptomes from victims deceased either due to non-infectious causes or due to COVID-19, was first analyzed for differentially expressed genes, as shown in FIG. 4A.

The DEGs are displayed as a heatmap labeled with selected genes in FIG. 4B. Bar plots displaying the ability of the differentially expressed genes in the test cohort (GSE151764) to classify human COVID-19 respiratory samples from four other independent cohorts is also shown in FIG. 4B.

A reactome-pathway analysis showing the major pathways up-regulated or down-regulated in the COVID-19-afflicted lungs is depicted in FIG. 4C and FIG. 4E. Differentially expressed genes showed an immunophenotype that was consistent with what is expected in viral infections and showed overrepresentation of pathways such as interferon, immune, and cytokine signaling. Differentially expressed gene signatures and reactome pathways that were enriched in the test cohort were fairly representative of the host immune response observed in patient-derived respiratory samples in multiple other validation cohorts. The signature derived from the test cohort could consistently classify control samples from COVID-19 samples.

Notably, the patient-derived signature was able to perfectly classify the EpCAM-sorted epithelial fractions from the bronchoalveolar lavage fluids of infected and healthy subjects (ROC AUC 1.00; GSE145926-Epithelium⁷⁴. This suggests that the respiratory epithelium is a major site where the host immune response is detected in COVID-19. When compared to existing organoid models of COVID-19, it was observed that the patient-derived COVID-19 lung signature was able to perfectly classify infected versus uninfected late passages (>50) of hiPSC-derived AT1/AT2 monolayers (GSE155241)⁵⁰ and infected versus uninfected liver and pancreatic organoids. The COVID-19-lung signatures failed to classify commonly used respiratory models, such as A549 cells and bronchial organoids, as well as intestinal organoids.

Similar data, depicting the ability of the differentially expressed genes in the test cohort (GSE151764) to classify published in vitro models for SARS-COV-2 infection where RNA sequence datasets were either generated in this work or publicly available, is shown in FIG. 4D.

A similar analysis of the lung models disclosed herein revealed that the COVID-19 lung signature was induced in submerged monolayers with distal-predominant AT2 to AT1 differentiation but not in the proximal-predominant ALI model. The ALI model and the small airway epithelia, both models that mimic the airway epithelia to mount the patient-derived immune signatures. These findings suggested that the presence of alveolar pneumocytes is critical for emulating host response. Surprisingly, induction of the COVID-19-lung signature also failed in hiPSC-derived AT2 monolayers, indicating that AT2 cells are unlikely to be the source of such host response. These findings indicate that both proximal airway and AT2 cells, when alone, are insufficient to induce the host immune response that is encountered in the lungs of COVID-19 patient.

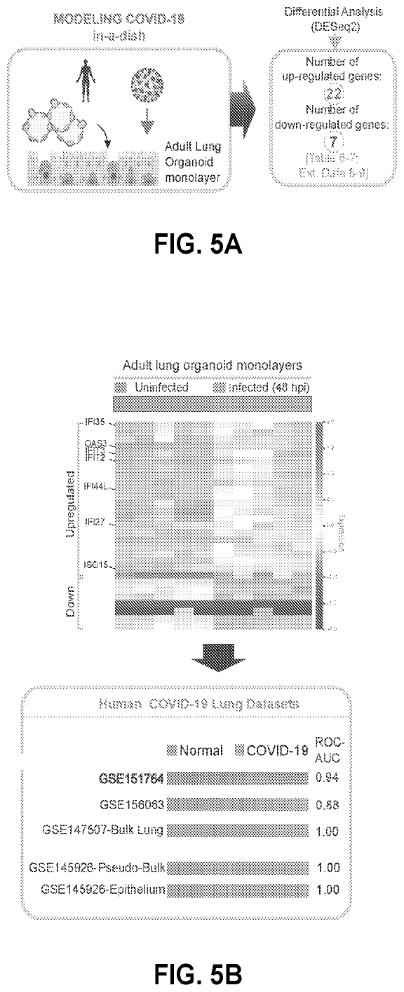

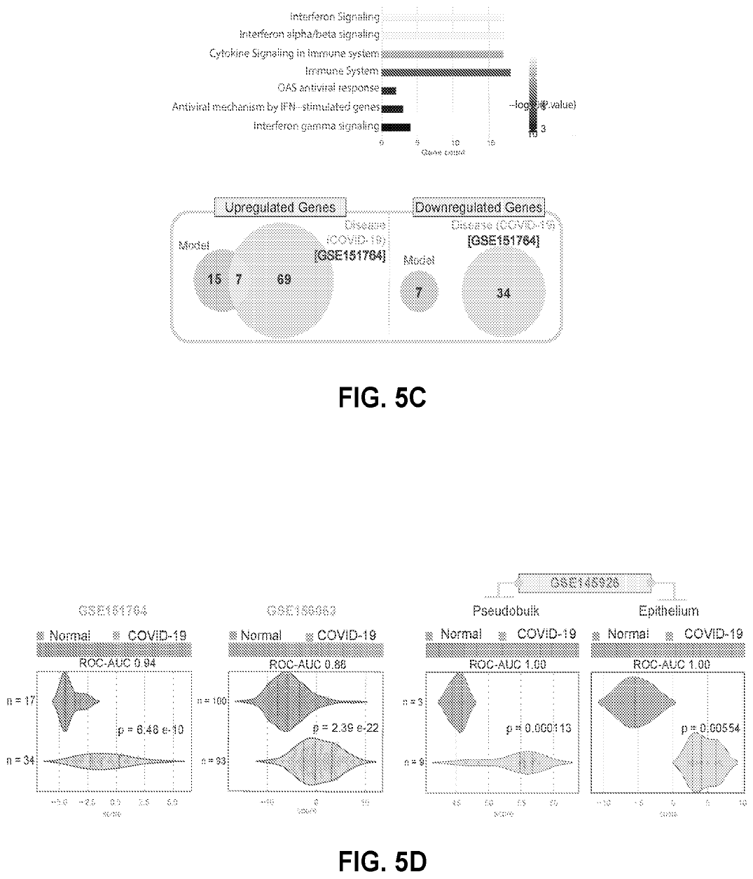

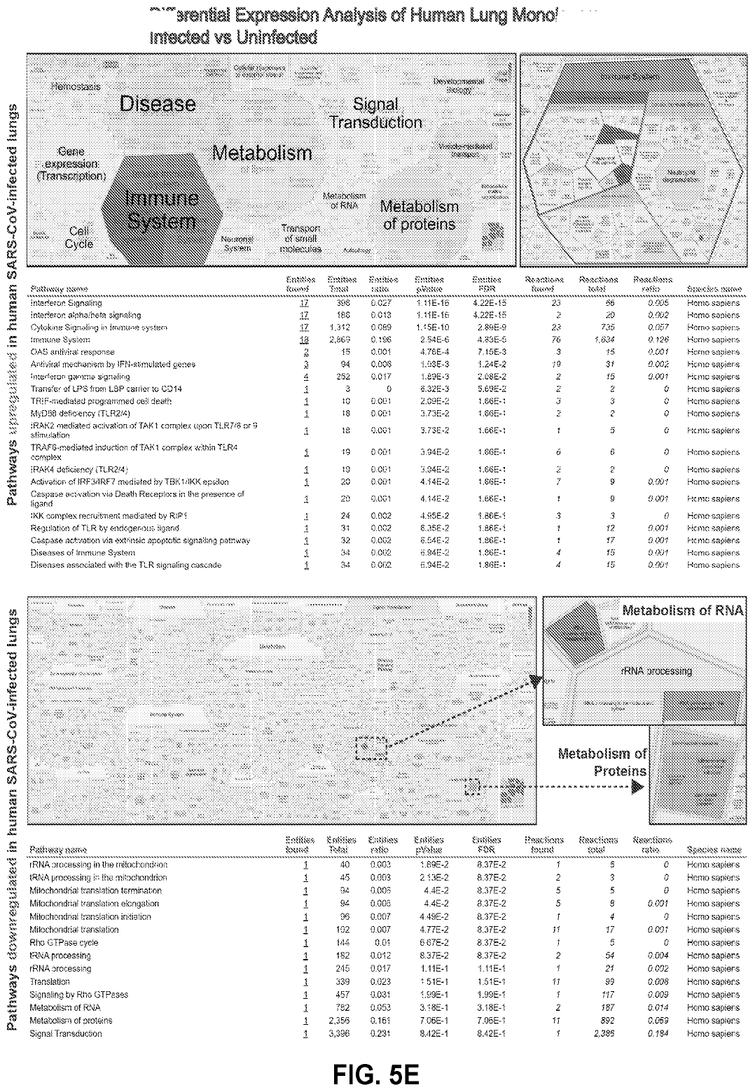

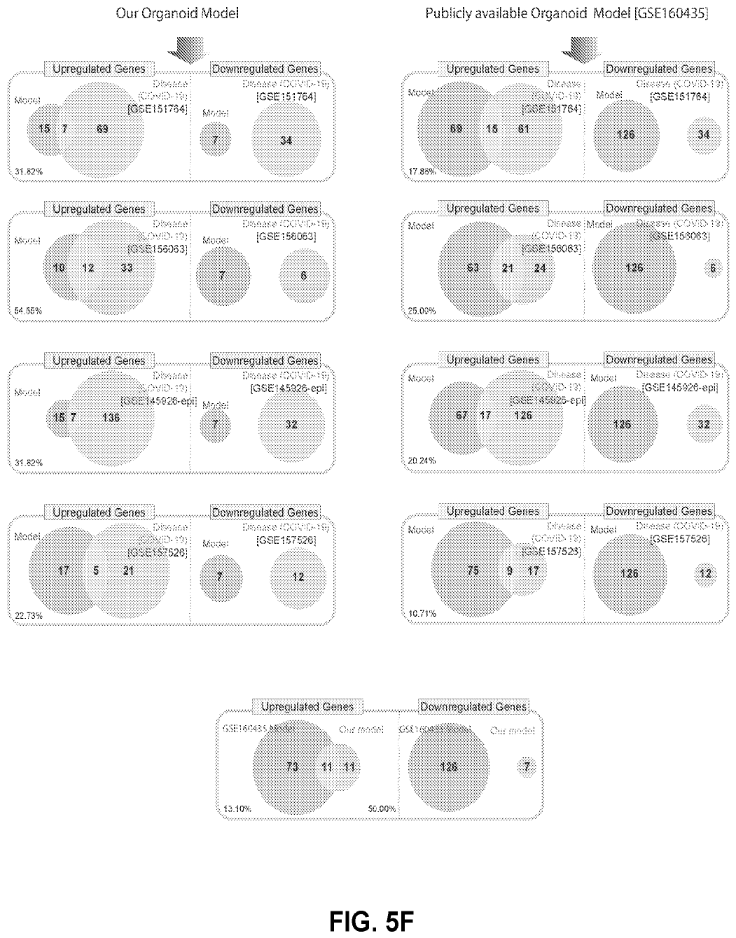

Next, datasets from the lung organoid monolayers for differentially expressed genes (DEGs) when challenged with SARS-COV-2, as shown in FIG. 5A. These differentially expressed genes are displayed as a heatmap in FIG. 5B. The ability of the DEGs in infected lung monolayers to classify human normal versus COVID-19 respiratory samples from five independent cohorts is also shown in FIG. 5B. A reactome-pathway analysis showing the major pathways upregulated in SARS-COV-2-infected lung organoid monolayers is shown in FIG. 5C and FIG. 5E. FIG. 5C also depicts the overlaps in DEGs between the model and disease.

Genes and pathways upregulated in the infected lung organoid-derived monolayer models overlapped significantly with those that were upregulated in the COVID-19 lung signature. It was observed only a partial overlap, ranging from ˜22-55% across various human datasets, in upregulated genes and no overlaps among downregulated genes between model and disease (COVID-19), as shown in FIG. 5C and FIG. 5F. Because the degree of overlap was even lesser, ranging from ˜10-25% across various human datasets, in the case of another publicly released model (GSE160435)¹¹, these discrepancies between the model and the actual disease likely reflect the missing stromal and immune components in our organoid monolayers.

Referring now to FIG. 5D, a comparison of the accuracy of disease modeling in three publicly available human lung datasets, as determined by the significant induction of the DEGs that were identified in the SARS-COV-2-challenged monolayers, is shown. Regardless of any missing components, the model-derived DEG signature was sufficient to consistently and accurately classify diverse cohorts of patient-derived respiratory samples. And the model-derived DEG signature was significantly induced in COVID-19 samples compared to normal controls. Most importantly, the model-derived DEG signature was significantly induced in the epithelial cells recovered from bronchoalveolar lavage.

Taken together, these cross-validation studies from disease to model, and vice versa, provide an objective assessment of the match between the host response in COVID-19 lungs and our submerged lung organoid monolayers. Such a match was not seen in the case of the other models, e.g., the proximal airway-mimic ALI model, HSAEpC monolayer, or hiPSC-derived AT2 models. Because the submerged ALO monolayers contained both proximal airway epithelia (basal cells) and promoted AT2 to AT1 differentiation, findings demonstrate that mixed cellular monolayers can mimic the host response in COVID-19. A subtractive analysis revealed that the cell type that is shared between models which showed induction of host response signatures, but is absent in models that do not show such response is AT1. Thus, the distal differentiation from AT2 to AT1, a complex process that is comprised of distinct intermediates⁷⁵, is essential for modeling the host immune response in COVID-19.

### Example 6: Both Proximal and Distal Airway Epithelia are Required to Mount the Overzealous Host Response in COVID-19

A determination of which model best simulated the overzealous host immune response that has been widely implicated in fatal COVID-19 was also analyzed. To this end, a recently described artificial intelligence (AI)-guided definition of the nature of the overzealous response in fatal COVID-19 was relied upon⁷⁶.

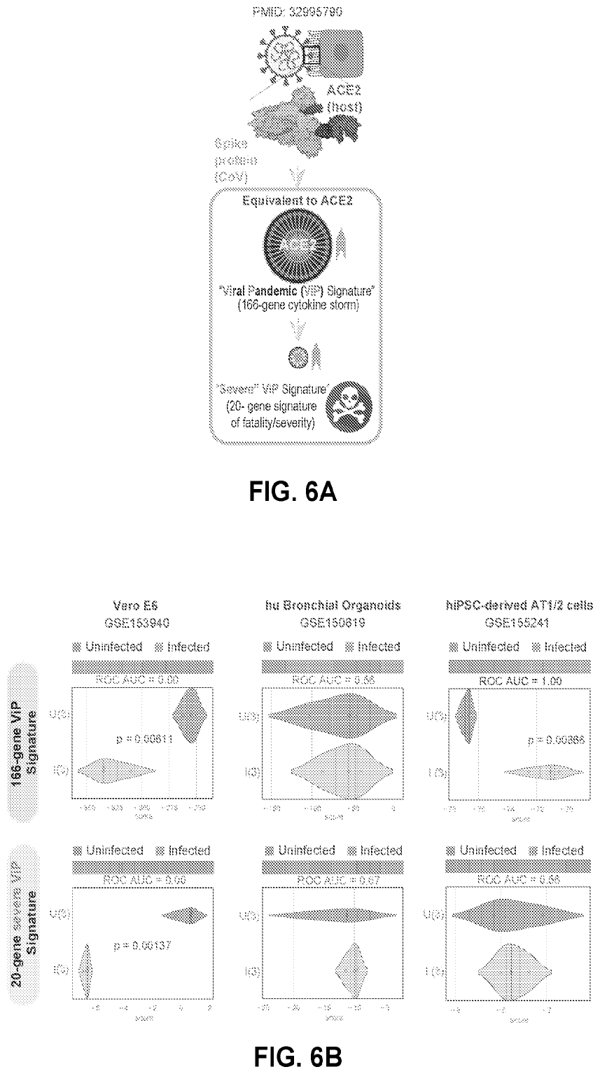

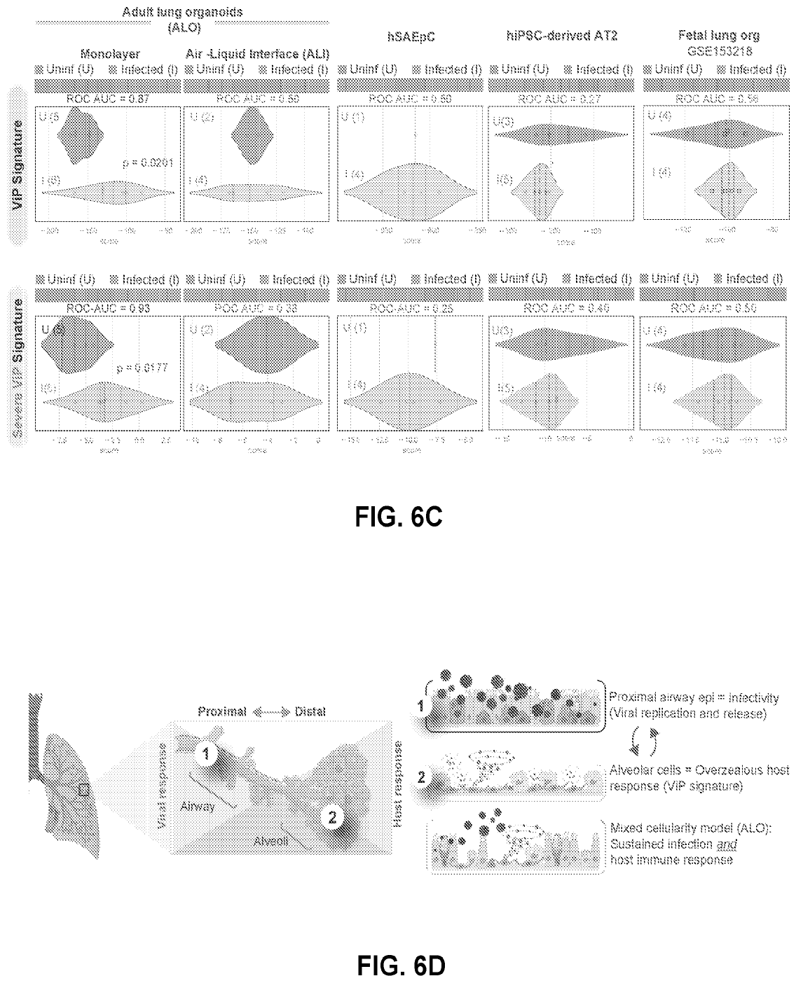

FIG. 6A depicts a schematic summarizing the immune signatures identified based on ACE2-equivalent gene induction observed invariably in any respiratory viral pandemic. The 166-gene ViP signature captures the cytokine storm in COVID-19, whereas the 20-gene subset severe ViP signature is indicative of disease severity/fatality. Using ACE2 as a seed gene, the 166-gene signature was identified and validated as an invariant immune response that was shared among all respiratory viral pandemics, including COVID-19. The subset of 20 genes within the 166-gene signature was subsequently identified as a determinant of disease severity/fatality. In particular, these 20 genes represented translational arrest, senescence, and apoptosis.

FIG. 6B depicts publicly available RNA sequence datasets from commonly used lung models. Vero E6, human bronchial organoids, and hPSC-derived AT1/AT2 cell-predominant lung organoids are classified using the 166-gene ViP signature and 20-gene severity signature. These two signatures, referred to as ViP (166-gene) and severe ViP (20-gene) signatures, were used as a computational framework to first validate existing SARS-COV-2 infection models that have been commonly used for therapeutic screens. Surprisingly, it was observed that each model fell short in one way or another. For example, the Vero E6, which is a commonly used cultured cell model, showed an opposite response of that which was anticipated. That is, instead of being induced, both the 166-gene and 20-gene ViP signatures were suppressed in infected Vero E6 monolayers. Similarly, neither ViP signature was induced in the case of SARS-COV-2 challenged human bronchial organoids¹⁶. Finally, in the case of the hiPSC-derived AT1/AT2 organoids, which recapitulated the COVID-19-lung derived immune signatures, the 166-gene ViP signature was induced significantly, but the 20-gene severity signature was not. These findings show that none of the existing models capture the overzealous host immune response that has been implicated in a fatality.

Turning to FIG. 6C, RNA sequence datasets generated in this work using either human small airway epithelial cells, adult lung organoids as submerged or ALI models and hiPSC-derived AT2 cells were analyzed.

The lung models showed that both the 166-gene and 20-gene ViP signatures were induced significantly in the submerged ALO-derived monolayers that had distal differentiation, but not in the proximal-mimic ALI model. Neither signatures were induced in monolayers of small airway epithelial cells or hiPSC-derived AT2 cells. For comparison, a published lung organoid model that that supports robust SARS-COV-2 infection was also analyzed. This model was generated using multipotent SOX2+SOX9+lung bud tip (LBT) progenitor cells that were isolated from the canalicular stage of human fetal lungs (˜16-17 wk post-conception)⁷⁷.

RNA sequence datasets from fetal lung organoid monolayers⁷⁷, infected or not with SARS-COV-2, were analyzed for the ability of ViP signatures to classify infected and uninfected samples. ROC AUC in all figure panels indicate the performance of a classification model using the ViP signatures. Despite mixed cellularity, this fetal lung organoid model failed to induce the ViP signatures⁷⁶. These findings indicate that despite having mixed cellular composition and the added advantage of being permissive to robust viral replication, the model lacks the signature host response that is seen in all human samples of COVID-19.

FIG. 6D depicts a summary of findings disclosed herein, and the relationship of these findings to the observed clinical phases in COVID-19. Taken together with our infectivity analyses, these findings demonstrate that although the proximal airway epithelia and AT2 cells may be infected, and as described by others^{21,37}, may be vital for mounting a viral response and for disease transmission, these cells alone cannot mount the overzealous host immune response that is associated with the fatal disease. Similarly, even though the alveolar pneumocytes, AT1 and AT2 cells, are sufficient to mount the host immune response, in the absence of proximal airway components, they too are insufficient to recapitulate the severe ViP signature that is characterized by cellular senescence and apoptosis. However, when both proximal and distal components are present, i.e., basal, ciliated and AT1 cells, the model mimicked the overzealous host immune response in COVID-19.

Notably, the adult lung organoids having both proximal airway and distal alveolar epithelia disclosed herein may not only be stably propagated and expanded in 3D cultures, but also may be used as monolayers of mixed cellularity for modeling viral and host immune responses during respiratory viral pandemics. Furthermore, an objective analysis of this model and other existing SARS-COV-2-infected lung models against patient-lung derived transcriptomes showed that the model which most closely emulates the elements of viral infectivity, lung injury, and inflammation in COVID-19 is one that contained both proximal and distal alveolar signatures, whereas the presence of just one or the other fell short.

The development of the human lung organoid models disclosed herein is significant in several ways. First and foremost, successful creation of adult human lung organoids having both proximal and distal signatures has not yet been accomplished. The multicellularity of the lung has been a daunting challenge that many experts have tried to recreate in vitro. While previous models have successfully used airway basal cells for organoid creation, models complete with other lung cells have been difficult to create⁷⁸. Moreover, the demand for perfecting such a multicellular model remains high, not just in the current context of the COVID-19 pandemic but also with potential of future pandemics.

Lung organoids created according to the methodology disclosed herein retain proximal and distal cellularity throughout multiple passages and even within a single organoid. Although a systematic design of experiment (DoE) approach⁷⁹ was not involved in developing the aforementioned lung organoids, a rationalized approach was taken. For example, a Wnt/R-spondin/Noggin-containing conditioned media was used as a source of the so-called ‘niche factors’ for any organoid growth⁸⁰. This was supplemented with recombinant FGF7, which is known to help cell proliferation and differentiation and required for normal branching morphogenesis⁸¹, and recombinant FGF10, which helps in cell maturation⁸² and alveolar regeneration upon injury.⁸³. Together, FGF7 and FGF10 are likely to direct differentiation toward distal lung lineages, and as such preserve alveolar signatures.

The presence of both distal alveolar and proximal ciliated cells is also significant. Proximal cells are effective to recreate sustained viral infectivity, whereas the distal alveolar pneumocytes, and in particular the ability of AT2 cells to differentiate into AT1 pneumocytes, are effective to recreate the host response to infection. One explanation for the criticality of these cells is that the viral response is mediated by a distinct AT2-lineage population-damage-associated transient progenitors (DATPs)—which arise as intermediates during AT2 to AT1 differentiation upon injury-induced alveolar regeneration⁷⁵. AT1 pneumocytes have also been documented to have mounted similar innate immune responses in bacterial pneumonia^{84,85}.

Other long-term ALI models using hiPSC-derived AT2 monolayers, in growth conditions that inhibit AT2 to AT1 differentiation, did show that SARS-COV-2 induces iAT2-intrinsic cytotoxicity and inflammatory response, but failed to induce type 1 interferon pathways (IFN α/β).⁵¹ Thus, prolonged culture of iAT2 pneumocytes may give rise to some DATPs but cannot robustly do so in the presence of inhibitors of AT1 differentiation. This spatially segregated viral and host immune response is common theme among lung infections, including bacterial pneumonia and other viral pandemics^{37,86-88}. As such, the mixed cellularity model is appropriate for use in modeling diverse lung infections and respiratory pandemics to come.

Second, among all the established lung models so far, the model disclosed herein features four key properties for rapid screening of candidate therapeutics and vaccines. These properties include (i) reproducibility, propagability and scalability, (ii) cost-effectiveness, (iii) personalization, and (iv) modularity, with the potential to add other immune and non-immune cell types to our multi-cellular complex lung model. The protocol disclosed herein has optimized support isolation, expansion and propagability in up to at least 12-15 passages, with documented retention of proximal and distal airway components up to P8 (by RNA seq). The models may also be scaled up and optimized for use in miniaturized 384-well infectivity assays. Protocols disclosed herein can also be reproduced across genders and without consideration of a donor's smoking status. That is, consistency in outcome and growth characteristics was observed across all isolation attempts.

The present lung organoids are also cost-effective, as the need for exclusive reliance on recombinant growth factors was replaced at least in part with conditioned media from a commonly used cell line (L-WRN/ATCC CRL-2647 cells). This conditioned media has a batch to batch stable cocktail of Wnt, R-Spondin, and Noggin, which has been shown to improve reproducibility in the context of GI organoids created in independent laboratories⁸⁹. Repeated iPSC-reprogramming has also been eliminated, further cutting costs compared to other existing models.

As for personalization, each organoid line, developed from adult lung stem cells from deep lung biopsies, was established from one patient. By avoiding iPSCs or EPSCs, this model not only captures genetics, but also retains organ-specific epigenetic programming. Additional programing, such as in the setting of chronic infection, injury, inflammation, or somatic mutations, may also occur. This ability to replicate donor phenotype and genotype in vitro allows for potential use as pre-clinical human models for Phase ‘0’ clinical trials.

Regarding modularity, by showing that the 3D lung organoids could be used as polarized monolayers on transwells to allow infectious agents to access the apical surface, such as SARS-COV-2, it is shown that the organoids have the potential to be reverse-engineered with additional components in a physiologically relevant spatially segregated manner. For example, immune and stromal cells can be placed in the lower chamber to model complex lung diseases that are yet to be modeled and have no cure, such as idiopathic pulmonary fibrosis.

Third, the value of the lung organoid models is further enhanced by the availability of companion readouts and biomarkers that can rapidly and objectively vet treatment efficacy based on set therapeutic goals. Companion readouts and biomarkers include, but are not limited to, ViP signatures in the case of respiratory viral pandemics, monitoring the E gene, or viral shedding. Of these readouts, the host response, as assessed by ViP signatures, is a key vantage point because an overzealous host response is what is known to cause fatality. Recently, a systematic review of the existing pre-clinical animal models revealed that most of the animal models of COVID-19 recapitulated mild patterns of human COVID-19. However, no severe illness associated with mortality was observed, suggesting a wide gap between COVID-19 in humans³⁸ and animal models⁹⁰. Notably, alternative models that effectively support viral replication, such as the proximal airway epithelium or iPSC-derived AT2 cells or a fetal lung bud tip-derived organoid model⁷⁷, also do not recapitulate the host response in COVID-19. The model disclosed herein, in conjunction with the ViP signatures described earlier⁷⁶, can serve as a pre-clinical model with companion diagnostics to identify drugs that target both the viral and host response in pandemics.

This model can be further improved by the simultaneous addition of endothelial cells and immune cells to better understand the pathophysiologic basis for DAD, microangiopathy, and even organizing fibrosis with loss of lung capacity that has been observed in many patients³⁸. These insights should be valuable to fight some of the long-term sequelae of COVID-19. Work with flow cytometry and cell sorting of our lung organoids will help understand each cell type's role in viral pathogenesis. Larger living biobanks of genotyped and phenotyped adult lung organoids representing donors of different age, ethnicity, predisposing conditions and co-existing comorbidities, will advance our understanding of why SARS-COV-2 and possibly other infectious agents may trigger different disease course in different hosts.

### Detailed Methods

### Collection of Human Lung Specimens for Organoid Isolation

To generate adult healthy lung organoids, a cardiothoracic surgeon resects a lung and thereafter collects fresh biopsy samples. Before collecting the lung specimens, each tissue was sent to a gross anatomy room where a pathologist cataloged the area of focus. Extra specimens were routed to the research lab in Human Transport Media (HTM, Advanced DMEM/F-12, 10 mM HEPES, 1× Glutamax, 1× penicillin-streptomycin, 5 μM Y-27632) for cell isolation. De-identified lung tissue deemed in excess by clinical pathologists are collected using an approved human research protocol (IRB #101590; PI: Thistlethwaite). Subsequently, organoids were isolated and biobanked from the excess lung tissue, a process which is carried out using an approved human research protocol (IRB #190105: PI Ghosh and Das) covering human subject research at the UC San Diego HUMANOID Center of Research Excellence (CoRE). A portion of the collected lung tissue specimen was fixed in 10% Zinc-Formalin for at least 24 hrs, then was submerged in 70% EtOH until being embedded in FFPE blocks.

For all the de-identified human subjects, information such as age, gender, and medical history was collected from the subject's chart. All information was collected in accordance with HIPAA rules.

Autopsy Procedures for Lung Tissue Collection from COVID-19 Positive Human Subjects

Lung specimens from COVID-19 positive human subjects were collected during autopsies (which was IRB exempt). Consent for tissue donations was obtained by telephone, followed by written email confirmation, by the next of kin or power of attorney, per California state law: Due to the COVID-19 pandemic, in-person visitation in the COVID-19 ICU was not permitted. Autopsies were performed in accordance with CDC COVID-19 guidelines and general autopsy procedures^{91,92}. Lung specimens were collected and stored in 10% Zinc-formalin for 72 hours before being processed for histology.

Autopsy #2 was a standard autopsy performed by anatomical pathology in the BSL3 autopsy suite. The patient expired and his family consented for autopsy. After 48 hours, the lungs were removed and immersion fixed whole in 10% formalin for 48 hours before being processed further. Lungs were only partially fixed at this time (about 50% fixed in thicker segments) and were sectioned into small 2-4 cm chunks and immersed in 10% formalin for further investigation.

Autopsy #4 and #5 were collected from rapid post-mortem lung biopsies. The procedure was performed in the Jacobs Medical Center ICU (all of the ICU rooms have a pressure-negative environment, with air exhausted through HEPA filters (Biosafety Level 3 (BSL3)) for isolation of SARS-COV-2 virus). Biopsies were performed 2-4 hours after patient expiration. The ventilator was shut off to reduce the aerosolization of viral particles at least 1 hour after the loss of pulse and before sample collection. Every team member had personal protective equipment in accordance with the University policies for procedures on patients with COVID-19 (N95 mask+surgical mask, hairnet, full face shield, surgical gowns, double surgical gloves, booties). Lung biopsies were obtained after L-thoracotomy in the 5th intercostal space by the cardiothoracic surgery team. Samples were taken from the left upper lobe (LUL) and left lower lobe (LLL) and then sectioned further.

### Isolation and Culture of Human Whole Lung-Derived Organoids

Existing scientific protocols were modified to isolate lung organoids from three human subjects^{31,33}. Normal human lung specimens were washed with PBS/4× penicillin-streptomycin and minced with surgical scissors. Tissue fragments were resuspended in 1.5 mL of wash buffer (Advanced DMEM/F-12, 10 mM HEPES, 1× Glutamax, 1× penicillin-streptomycin) containing 2 mg/ml Collagenase Type I (Thermo Fisher, USA) and incubated at 37° C. for approximately 1 hour. During incubation, tissue pieces were sheared every 10 minutes with a 1 mL micropipette and examined under a light microscope to monitor the progress of digestion. When 80-100% of single cells were released from connective tissue, the digestion buffer was neutralized with 10 mL wash buffer with added 2% Fetal Bovine Serum. This suspension was then passed through a 100-μm cell strainer and centrifuged at 200 rcf. Remaining erythrocytes were lysed in 2 ml red blood cell lysis buffer (Invitrogen) at room temperature for 5 minutes, followed by the addition of 10 mL of wash buffer and centrifugation at 200 rcf.

Cell pellets were resuspended in cold Matrigel (Corning, USA) and seeded in 25 μl droplets on a 12 well tissue culture plate. The plate was inverted and incubated at 37° C. for 10 minutes to allow complete polymerization of the Matrigel before the addition of 1 mL Lungbrew media¹²⁰ (Complete Medium Composition in the chart below) per well. Lungbrew media was prepared by modifying a media that was previously optimized for growing gastrointestinal (GI)-organoids (50% conditioned media, prepared from L-WRN cells with Wnt3a, R-spondin, and Noggin, ATCC-CRL-3276).^{42,93-95}.

The lung expansion media was compared to alveolosphere media I (IMDM and F12 as the basal medium with B27, low concentration of KGF, Monothioglycerol, GSK3 inhibitor, Ascorbic acid, Dexamethasone, IBMX, cAMP and ROCK inhibitor) and II (F12 as the basal medium with added CaCl₂), B27, low concentration of KGF, GSK3 inhibitor, TGF-β receptor inhibitor Dexamethasone, IBMX, cAMP and ROCK inhibitor) modified from previously published literature^{27,28}. Neither alvelosphere media contain any added Wnt3a, R-spondin, and Noggin. The composition of these media was developed either by fundamentals of adult-stem cell-derived mixed epithelial cellularity in other organs (like the gastrointestinal tract⁴⁰⁻⁴², or rationalized based on published growth conditions for proximal and distal airway components^{25,31,32}.

Organoids were maintained in a humidified incubator at 37° C./5% CO₂, with a complete media change performed every 3 days. After the organoids reached confluency between 7-10 days, organoids were collected in PBS/0.5 mM EDTA and centrifuged at 200 rcf for 5 min. Organoids were dissociated in 1 mL trypLE Select (Gibco, USA) per well at 37° C. for 4-5 min and mechanically sheared. Wash buffer was added at a 1:5, trypLE to wash buffer ratio. The cell suspension was subsequently centrifuged, resuspended in Matrigel, and seeded at a 1:5 ratio. Lung organoids were biobanked and passage 3-8 cells were used for experiments. Subculture was performed every 7-10 days.

After isolation, organoids were monitored for growth and cell health for 4-7 days. The organoids were then split and maintained in a humidified incubator at 37° ° C./5% CO₂. A complete media change was performed every 3 days. Organoids reached confluency between 7-10 days after isolation. Organoids were then scraped and collected in PBS/0.5 mM EDTA and centrifuged at 200 rcf for 5 minutes. Organoids were dissociated in 2 mL trypLE Select (Gibco, USA) per 12 wells of organoids at 37° C. for 4-5 minutes, and mechanically sheared by pipetting up and down 7-10 times, or until visible clumps were gone or minimally present. Wash buffer was added at a 1-to-5 trypLE to wash buffer ratio. The cell suspension was subsequently centrifuged, resuspended in Matrigel, and seeded at a 1-to-5 ratio. Lung organoids were biobanked and passage 3-8 cells were used for experiments. Subcultures were performed every 7-10 days.

TABLE 1
Lungbrew Media

| | Company: | | | Stock | | | |
|---|---|---|---|---|---|---|---|
| Components | Catalog # | Stock Conc | | volume | | Final conc | |
| 50% GI CM | | | | 48 | ml | | |
| Advanced | Gibco: | | | 48 | ml | | |
| DMEM-F12 | 12634-010 | | | | | | |
| HEPES | Gibco: | 1M | | 1 | ml | 10 | mM |
| | 15630-080 | | | | | | |
| Glutamax | Gibco: | 200 | mM | 1 | ml | 2 | mM |
| | 35050-061 | | | | | | |
| B27 | Gibco: | 50X | | 1 | ml | 0.5X | |
| Supplement | 17504-044 | | | | | | |
| N-acetyl | Sigma: A9165 | 500 | mM | 250 | ul | 1.25 | mM |
| cysteine | | | | | | | |
| Nicotinamide | Sigma: N0636 | 1M | | 500 | ul | 5 | mM |
| FGF7 (KGF) | PeproTech: | 100 | ug/ml | 25 | ul | 25 | ng/ml |
| | 100-19 | | | | | | |
| FGF10 | PeproTech: | 100 | ug/ml | 100 | ul | 100 | ng/ml |
| | 100-26 | | | | | | |
| A-83-01 | Tocris | 2 | mM | 25 | ul | 500 | nM |
| | (R&D): 2939 | | | | | | |
| SB202190 | Sigma: S7067 | 30 | mM | 34 | ul | 10 | uM |
| Pen Strep | Gibco: | 100X | | 1 | ml | 1X | |
| | 15140-122 | | | | | | |
| Y-27632 | Tocris | 10 | mM | 50 | ul | 5 | uM |
| | (R&D): 1254 | | | | | | |

### The Preparation of Lung Organoid-Derived Monolayers

Lung-organoid-derived monolayers were prepared using a modified protocol of GI-organoid-derived monolayers^{42,93-95}. Transwell inserts (6.5 mm diameter, 0.4 μm pore size, Corning) were coated in Matrigel, diluted in cold PBS at a 1-to-40 ratio, and incubated for 1 hour at room temperature. Confluent organoids were collected in PBS/EDTA after 7 days and dissociated into single cells in trypLE for 6-7 minutes at 37° ° C. Following enzymatic digestion, the cell suspension was mechanically sheared through vigorous pipetting with a 1000 μl pipette and neutralized with wash buffer. The suspension was centrifuged, resuspended in Pneumacult Ex-Plus Medium (StemCell, Canada), and passed through a 70-μm cell strainer. The coating solution was aspirated and cells were seeded onto the apical membrane at 1.8E5 cells per transwell with 200 μl PneumaCult Ex-Plus media. 700 μl of PneumaCult Ex-Plus was added to the basal chamber and cells were cultured over the course of 2-4 days. Media changes for both the apical and basal chambers was performed every 24 hours.

### The Preparation of Lung Organoid-Derived Monolayers for Infection and Functional Assays

Densely packed organoids, collected in PBS/0.5 mM EDTA were centrifuged at 200 rcf for 5 minutes. Organoids were dissociated in 2 mL trypLE Select (Gibco, USA) per 12 wells of organoids at 37ºC for 4-5 minutes and mechanically sheared by pipetting up and down. The TrypLE was inactivated with al-to-5 trypLE to Lung Wash Media ratio. The cells were thereafter centrifuged at 300 g for 4 minutes. The supernatant was aspirated and resuspended in the cell pellet at room temperature using PneumaCult Ex-Plus Mix, with larger clumps of cells being filtered out using 70 μM filter. The viable cells were counted and plated using PneumaCult Ex-Plus media. Cells were either plated 105 cells per well in a 96 well plate, or 2×10⁴ cells per well in a 384 well Matrigel coated plate. The media was changed after 24 hours of incubation. After 48 hours of plating, the cells were differentiated and appeared flattened. The cells were then infected with MOI of 0.5 with SARS-COV-2. After treatment, the lung monolayers were used for RNA sequencing, immunostaining, and other functional assay.

### Human Whole Lung-Derived Organoids in Fibrosis Modeling

Organoids were cultured in Lungbrew media as described above for 5 days with a complete media change every 2 days. On the fifth day of organoid culture, media was changed to Lungbrew-fibrosis media, with or without 1 μM of Thapsigarign (Sigma-Aldrich), and incubated for 16 hours to induce ER stress. After incubation with Thapsigargin, the media was changed to Lungbrew-fibrosis media only, and thereafter was changed every 2 days. The organoids were collected on day 12 in Cell Recovery Solution (Corning) and profibrotic mRNA transcripts were measured via RT-qPCR.

TABLE 2
Lungbrew Fibrosis Media

| | Company: | | | Stock | | | |
|---|---|---|---|---|---|---|---|
| Components | Catalog # | Stock Conc | | volume | | Final conc | |
| 50% GI CM | | | | 48 | ml | | |
| Advanced | Gibco: | | | 48 | ml | | |
| DMEM-F12 | 12634-010 | | | | | | |
| HEPES | Gibco: | 1M | | 1 | ml | 10 | mM |
| | 15630-080 | | | | | | |
| Glutamax | Gibco: | 200 | mM | 1 | ml | 2 | mM |
| | 35050-061 | | | | | | |
| B27 Supplement | Gibco: | 50X | | 1 | ml | 0.5X | |
| | 17504-044 | | | | | | |
| N-acetyl cysteine | Sigma: A9165 | 500 | mM | 200 | ul | 1 | mM |
| Nicotinamide | Sigma: N0636 | 1M | | 100 | ul | 1 | mM |
| FGF7 (KGF) | PeproTech: | 100 | ug/ml | 15 | ul | 15 | ng/ml |
| | 100-19 | | | | | | |
| FGF10 | PeproTech: | 100 | ug/ml | 50 | ul | 50 | ng/ml |
| | 100-26 | | | | | | |
| Pen Strep | Gibco: | 100X | | 1 | ml | 1X | |
| | 15140-122 | | | | | | |
| Y-27632 | Tocris | 10 | mM | 25 | ul | 2.5 | uM |
| | (R&D): 1254 | | | | | | |

### Air-Liquid Interface (ALI) Model of Lung Organoids

Organoids were dissociated into single cells and expanded in T-75 flasks in PneumaCult Ex-Plus Medium until confluency was reached. Cells were dissociated in ACF Enzymatic Dissociation Solution (StemCell, Canada) for 6-7 minutes at 37° C. and neutralized in equal volume ACF Enzyme Inhibition Solution (StemCell, Canada). Cells were seeded in the apical chamber of transwells at 3.3E4 cells per transwell in 200 μL of PneumaCult Ex-Plus Medium. 500 μL of PneumaCult Ex-Plus was added to the basal chamber. Media in both chambers was changed every other day until confluency was reached in approximately 4 days.

When confluency was reached, the media was completely removed from the apical chamber, and media in the basal chamber was replaced with ALI Maintenance Medium (StemCell, Canada). The media in the basal chamber was changed every 2 days. The apical chamber was washed with warm PBS every 5-7 days to remove accumulated mucus. Cells were cultured under ALI conditions for at least 21 days until they completed differentiation into a pseudostratified mucociliary epithelium. To assess the integrity of the epithelial barrier, Trans-Epithelial Electrical Resistance (TEER) was measured with an Epithelial Volt-Ohm Meter (Millicell, USA). The media was removed from the basal chamber, and wash media was added to both chambers. Cultures were equilibrated to 37° C. before TEER values were measured. Final values were expressed as Ω·cm² units and were calculated by multiplying the growth area of the membrane by the raw TEER value.

The Culture of Primary Airway Epithelial Cells and iPSC-Derived Alveolar Epithelial Cells

Primary normal human bronchial epithelial cells (NHBE) were obtained from Lonza and grown according to instructions. NHBE cells were cultured in T25 cell culture tissue flasks with PneumaCult Ex-Plus media (StemCell, Canada). Cells were seeded at approximately 100,000 cells/T25 flask and incubated at 37° C., 5% CO₂. Once cells reached 70-80% confluency, they were dissociated using 0.25% Trypsin in dissociation media and plated in 24 well transwells (Corning). Primary human bronchial epithelial cells (HBEpC) and small airway epithelial cells (HSAEpC) were obtained from Cell Applications Inc. The HBEpC and HSAEpC were cultured in human bronchial/tracheal epithelial cell media and small airway epithelial cell media, respectively, following the instructions of Cell Application.

Human iPSC-derived alveolar epithelial type 2 cells (iHAEpC2) were obtained from Cell Applications Inc. and cultured in growth media (1536K-05, Cell Applications Inc.), according to the manufacturer's instructions. All the primary cells were used within early passages (5-6) to avoid any gradual disintegration of the airway epithelium with columnar epithelial structure and epithelial integrity.

Infection with SARS-COV-2

Lung organoid-derived monolayers or primary airway epithelial cells, either in 384 well plates or in transwells, were washed twice with antibiotic-free lung wash media. 1E5 PFU of SARS-COV-2 strain USA-WA1/2020 (BEI Resources NR-52281) in complete DMEM was added to the apical side of the transwell and allowed to incubate for 24, 48, 72 and 96 hours at 34° C. and 5% CO₂. After incubation, the media was removed from the basal side of the transwell. The apical side of the transwells was then washed twice with antibiotic-free lung wash media, and then twice with PBS. TRIzol Reagent (Thermo Fisher 15596026) was then added to the well and incubated at 34° C. and 5% CO₂ for 10 minutes. After incubation, the TRIzol Reagent was removed and stored at −80° C. for RNA analysis.

### RNA Isolation

Organoids and monolayers used for lung cell type studies were lysed using RNA lysis buffer, followed by RNA extraction per the Zymo Research Quick-RNA MicroPrep Kit instructions. Tissue samples and monolayers in SARS-COV-2 studies were lysed in TRI-Reagent and RNA was extracted using the Zymo Research Direct-zol RNA Miniprep Kit.

Quantitative (q)RT-PCR

Organoid and monolayer cell-type gene expression was measured by qRT-PCR using 2×SYBR Green qPCR Master Mix. cDNA was amplified with gene-specific primer/probe set for the lung cell type markers and qScript cDNA SuperMix (5×). qRT-PCR was performed with the Applied Biosystems QuantStudio 5 Real-Time PCR System. Cycling parameters were as follows: 95° ° C. for 20 seconds, followed by 40 cycles of 1 second at 95° C. and 20 seconds at 60° ° C. All samples were assayed in triplicate and eukaryotic 18S ribosomal RNA was used as a reference.

| Cell types | Marker | Primer Sequence |
|---|---|---|
| Basal cells | ITGA6 | ITGA6 F |
| | | ‘CGAAACCAAGGTTCTGAGCCC’ |
| | | ITGA6 R |
| | | ‘CTTGGATCTCCACTGAGGCAGT’ |
| Goblet | Muc5AC | Muc5AC F |
| | | ‘GGAACTGTGGGGACAGCTCTT’ |
| | | Muc5ACR |
| | | ‘GTCACATTCCTCAGCGAGGTC’ |
| Cilia | FoxJ1 | FoxJ1 F |
| | | ‘ACTCGTATGCCACGCTCATCTG’ |
| | | FoxJ1 R |
| | | ‘GAGACAGGTTGTGGCGGATTGA’ |
| Club cell | SCGB1A1 | SCGB1A1 F |
| | | ‘CAAAAGCCCAGAGAAAGCATC’ |
| | | SCGB1A1 R |
| | | ‘CAGTTGGGGATCTTCAGCTTC’ |
| Alveolar | AQP5, PDPN | AQP5 F |
| type 1 | | ‘TACGGTGTGGCACCGCTCAATG’ |
| | | AQP5 R |
| | | ‘AGTCAGTGGAGGCGAAGATGCA’ |
| | | PDPN F |
| | | ‘GTGCCGAAGATGATGTGGTGAC’ |
| | | PDPN R |
| | | ‘GGACTGTGCTTTCTGAAGTTGGC’ |
| Alveolar | SFTPA1, | SFTPA1 F |
| type 2 | SFTPC | ‘CACCTGGAGAAATGCCATGTCC’ |
| | | SFTPAI R |
| | | ‘AAGTCGTGGAGTGTGGCTTGGA’ |
| | | SFTPC F |
| | | ‘GTCCTCATCGTCGTGGTGATTG’ |
| | | SFTPC R |
| | | ‘AGAAGGTGGCAGTGGTAACCAG’ |
| Stem Cell | NGFR, TP63 | NGFR F’ |
| | | CCTCATCCCTGTCTATTGCTCC |
| | | NGFR R’ |
| | | GTTGGCTCCTTGCTTGTTCTGC |
| | | TP63 F’ |
| | | CAGGAAGACAGAGTGTGCTGGT |
| | | TP63 R’ |
| | | AATTGGACGGCGGTTCATCCCT |
| Fibroblast | Col-1α, | Col-1a F’ |
| | αSMA, | CAAGAGGAAGGCCAAGTCGAGG |
| | | Col-1a R’ |
| | | CGTTGTCGCAGACGCAGAT |
| | | aSMA F’ |
| | | ACTGAGCGTGGCTATTCCTCCGTT |
| | | aSMA R’ |
| | | GCAGTGGCCATCTCATTTTCA |
| | | PDGFRA F’ |
| | | GACTTTCGCCAAAGTGGAGGAG |
| | | PDGFRA R’ |
| | PDGFRA | AGCCACCGTGAGTTCAGAACGC |

### Assessment of SARS-COV-2 Infectivity Test

Assessment of SARS-COV-2 infectivity test was determined by qPCR using TaqMan assays and TaqMan Universal PCR Master Mix96.97. cDNA was amplified with gene-specific primer/probe set for the E gene and qPCR was performed with the Applied Biosystems QuantStudio 3 Real-Time PCR System. The specific TaqMan primer/probe set for E gene as follows: Forward 5′- are ACAGGTACGTTAATAGTTAATAGCGT-3′, (IDT, Cat #10006888); Reverse 5′-ATATTGCAGCAGTACGCACACA-3″: Probe 5′-FAM-ACACTAGCCATCCTTACTGCGCTTCG-BBQ-3′ and 18S rRNA. Cycling parameters were as follows: 95ºC for 20 seconds, followed by 40 cycles of 1 second at 95° C. and 20 seconds at 60° C. All samples were assayed in triplicate and gene eukaryotic 18S ribosomal RNA was used as a reference.

### Immunofluorescence

Organoids and lung organoid-derived monolayers were fixed by either: (1) 4% PFA at room temperature for 30 minutes followed by quenching with 30 mM glycine for 5 minutes, (2) ice-cold 100% Methanol at −20° ° C. for 20 minutes, or (3) ice-cold 100% Methanol on ice for 20 minutes. Subsequently, samples were permeabilized and blocked for 2 hours using an in-house blocking buffer (2 mg/mL BSA and 0.1% Triton X-100 in PBS)⁹⁸. Primary antibodies were diluted in blocking buffer and allowed to incubate overnight at 4° C. Secondary antibodies were diluted in blocking buffer and allowed to incubate for 2 hours in the dark. ProLong Glass was used as a mounting medium, and #1 Thick Coverslips were applied to slides and sealed. Samples were stored at 4° C. until imaged.

FFPE embedded Organoid and Lung Tissue sections underwent antigen retrieval, as previously described in methods for Immunohistochemistry staining. After antigen retrieval and cooling in DI water, samples were permeabilized and blocked in blocking buffer and treated, as mentioned above for immunofluorescence. Images were acquired at room temperature with Leica TCS SPE confocal and with DMI4000 B microscope using the Leica LAS-AF Software. Images were taken with a 40× oil-immersion objective using 405-, 488-, 561-nm laser lines for excitation. Z-stack images were acquired by successive Z-slices of 1 μm in the desired confocal channels. Fields of view that were representative and/or of interest were determined by randomly imaging three different fields. Z-slices of a Z-stack were overlaid to create maximum intensity projection images: all images were processed using FIJI (Image J) software.

### Embedding of Organoids in HistoGel

Organoids were seeded on a layer of Matrigel in 6-well plates and grown for 7-8 days. Once mature, organoids were fixed in 4% PFA at room temperature for 30 minutes, followed by quenching with 30 mM glycine for 5 minutes. Organoids were gently washed with PBS and harvested using a cell scraper. Organoids were resuspended in PBS using wide-bore 1000 μL pipette tips. Organoids were stained using Gill's hematoxylin for 5 minutes for easier FFPE embedding and sectioning visualization. Hematoxylin stained organoids were gently washed in PBS and centrifuged and excess hematoxylin was aspirated. Organoids were resuspended in 65° C. HistoGel and centrifuged at 65° ° C. for 5 minutes. The HistoGel embedded organoid pellets were allowed to cool to room temperature, and then were stored in 70% ethanol at 4° C. until the organoid pellets were ready for FFPE embedding by LJI Histology Core. Successive FFPE embedded organoid sections were cut at a 4 μm thickness and fixed on to microscope slides.

### Immunohistochemistry

For SARS-COV-2 nucleoprotein (np) antigen retrieval, slides were immersed in Tris-EDTA buffer (pH 9.0) and boiled for 10 minutes at 100° ° C. inside a pressure cooker. Endogenous peroxidase activity was blocked by incubation with 3% H₂O₂ for 10 minutes. To block non-specific protein binding, 2.5% goat serum was added. The slides were then incubated with a rabbit SARS-COV-NP antibody (Sino Biological) for 1.5 hours at room temperature in a humidified chamber, then rinsed with TBS or PBS three times for 5 minutes each. Sections were also incubated with horse anti-rabbit IgG secondary antibodies for 30 minutes at room temperature, then washed with TBS or PBS three times for 5 minutes each. Then the sections were incubated with DAB and counterstained with hematoxylin for 30 seconds.

### Permeability of Lung Monolayer Using FITC-Dextran

Adult lung monolayers were grown for 2 days in PneumaCult Ex-Plus media on transwell inserts (6.5 mm diameter, 0.4 μm pore size, Corning). Trans-Epithelial Electrical Resistance (TEER) was monitored with an Epithelial Volt-Ohm Meter (Millicell, USA). On the second day of growth, FITC-dextran (10 kD) was added at a 1-to-50 dilution in lung wash media and the basolateral side of the insert was changed to lung wash media only. After 30 minutes of incubation with FITC-dextran, 50 μl of the basolateral supernatant was transferred to an opaque welled 96-well plate. Fluorescence was measured using a TECAN plate reader.

### The Characterization of Lung Cell Types Using Flow Cytometry

Lung organoids were dissociated into single cells via trypLE digestion and strained with a 30 μm filter (Miltenyi Biotec, Germany). Approximately 2.5E5 cells for each sample were fixed and permeabilized at room temperature in Cyto-Fast Fix Perm buffer (BioLegend, USA) for 20 minutes. The samples were subsequently washed with Cyto-Fast Perm Wash solution (BioLegend, USA) and incubated with lung epithelial cell type markers for 30 minutes. Following primary antibody incubation, the samples were washed and incubated with Propidium Iodide (Invitrogen) and Alexa Flour 488 secondary antibodies (Invitrogen) for 30 minutes. Samples were then resuspended in FACS buffer (PBS, 5% FBS, 2 mM Sodium Azide). Flow cytometry was performed using Guava easy Cyte benchtop flow cytometer (Millipore) and data was analyzed using InCyte (version 3.3) and FlowJo X v10 software.

### RNA Sequencing

RNA sequencing libraries were generated using the Illumina TruSeq Stranded Total RNA Library Prep Gold with TruSeq Unique Dual Indexes (Illumina, San Diego, CA). Samples were generally processed according to the manufacturer's instructions, except the RNA shear time were modified to five minutes. The resulting libraries were multiplexed and sequenced with 100 basepair (bp) Paired-End (PE100) to a depth of approximately 25-40 million reads per sample on an Illumina NovaSeq 6000 by the Institute of Genomic Medicine (IGM) at the University of California San Diego. Samples were demultiplexed using a bcl2fastq v2.20 Conversion Software (Illumina, San Diego, CA). RNA sequence data was processed using kallisto (version 0.45.0) and human genome GRCh38 Ensembl version 94 annotation (Homo sapiens GRCh38.94 chr_patch_hapl_scaff.gtf). Gene-level TPM values and gene annotations were computed using tximport and biomaRt R package. A custom python script was used to organize the data, which was log reduced using log 2(TPM+1). The raw data and processed data are deposited in Gene Expression Omnibus under accession numbers GSE157055 and GSE157057.

### Data Collection and Annotation

Publicly available COVID-19 gene expression databases were downloaded from the National Center for Biotechnology Information (NCBI) Gene Expression Omnibus website (GEO)⁹⁹⁻¹⁰¹. RMA (Robust Multichip Average)^{102,103} was used for microarrays where the dataset was not normalized, and TPM (Transcripts Per Millions)^{104,105} was used for RNA sequence data for normalization. We used log 2(TPM+1) to compute the final log-reduced expression values for RNA sequence data. All of the above datasets were processed using the Hegemon data analysis framework¹⁰⁶⁻¹⁰⁸

### Analysis of RNA Sequence Datasets

DESeq2¹⁰⁹ was applied to both uninfected and infected samples to identify up-regulated and down-regulated genes. A gene signature score was computed using both the up-regulated and down-regulated genes, which was then used to order the samples. To compute the gene signature score, the genes present in this list were first normalized according to a modified Z-score approach centered around StepMiner threshold (formula=(expr−Thr)/3*stddev). The normalized expression values for every probeset for all the genes were added or subtracted (depending on up-regulated and down-regulated genes) to create the final score. The Gene signature score is used to classify sample categories and the performance of the multi-class classification is measured by ROC-AUC (Receiver Operating Characteristics Area Under the Curve) values.

A color-coded bar plot is combined with a violin plot to visualize the gene signature-based classification. All statistical tests were performed using R version 3.2.3 (2015-12-10). Standard t-tests were performed using python scipy.stats.ttest_ind package (version 0.19.0) with Welch's Two Sample t-test (unpaired, unequal variance (equal_var=False), and unequal sample size) parameters. Multiple hypothesis correction was performed by adjusting p-values with statsmodels.stats.multitest.multipletests (fdr_bh: Benjamini/Hochberg principles). The results were independently validated with R statistical software (R version 3.6.1: 2019-07-05). Pathway analysis of gene lists was carried out via the Reactome database and algorithm¹¹⁰. Reactome identifies signaling and metabolic molecules and organizes their relations into biological pathways and processes. Violin, Swarm and Bubble plots were created using the Python Seaborn Package version 0.10.1.

### Single Cell RNA Sequence Data Analysis

Single Cell RNA sequence data from GSE145926 was downloaded from GEO in the HDF5 Feature Barcode Matrix Format. The filtered barcode data matrix was processed using the Seurat v3 R packagelll and Scanpy Python package¹¹². Pseudo bulk analysis of GSE145926 data was performed by adding counts from the different cell subtypes and normalized using log 2(CPM+1). Epithelial cells were identified with SFTPA1, SFTPB, AGER, AQP4, SFTPC, SCGB3A2, KRT5, CYP2F1, CCDCl53, and TPPP3 genes using the SCINA algorithm¹¹³. Pseudo bulk datasets were prepared by adding counts from the selected cells and normalized using log (CPM+1).

### Assessment of Cell-Type Proportions

CIBERSORTx (https://cibersortx.stanford.edu/runcibersortx.php) was used for cell-type deconvolution of the collected date, which was normalized by CPM. The Single Cell RNA sequence dataset (GSE132914) from Gene Expression Omnibus (GEO) was used as a reference. Next, the bulk RNA sequence3 datasets were analyzed to identify the cell types of interest using relevant gene markers. These gene markers include PDPN, AQP5, P2RX4, TIMP3, and SERPINE1 for AT1 cells: SFTPA1, SFTPB, SFTPC, SFTPD, SCGB1A1, ABCA3, and LAMP3 for AT2 cells: CD44, KRT5, KRT13, KRT14, CKAP4, NGFR, and ITGA6 for basal cells: SCGB1A1, SCGB3A2, SFTPA1, SFTPB, SFTPD, ITGA6, and CYP2F1 for club cells: CDX2, MUC5AC, MUC5B, and TFF3 for goblet cells: ACTG2, TUBB4A, FOXA3, FOXJ1, and SNTN for ciliated cells; and GJA1, TTF1, and EPCAM for Generic Lung Lineage cells. The relevant gene markers were identified using the SCINA algorithm. Then, normalized pseudo counts were obtained with the CPM normalization method. The cell-type signature matrix was derived from the Single Cell RNA sequence dataset, cell-types, and gene markers of interest. This matrix was constructed by taking an average from gene expression levels for each of the markers across each cell type.

### Statistical Analysis

All experiments were repeated at least three times, and results were presented as a representative experiment or an average #S.E.M. Statistical significance between datasets with three or more experimental groups was determined using one-way ANOVA, including a Tukey's test for multiple comparisons. For all tests, a P-value of 0.05 was used as the cutoff to determine significance (*P<0.05, ** P<0.01, *** P<0.001, and **** P<0.0001). All statistical analyses were performed using GraphPad prism 6.1. Some of the statistical tests were performed using R version 3.2.3 (2015-12-10). Standard t-tests were performed using Python scipy.stats.ttest_ind package (version 0.19.0).

### REFERENCES

1. Arrossi, A. V. & Farver, C. The pulmonary pathology of COVID-19. Cleve Clin J Med, doi: 10.3949/ccjm.87a.ccc063 (2020).
2. Damiani, S. et al. Pathological Post Mortem Findings in Lungs Infected With Sars-Cov 2. J Pathol, doi: 10.1002/path.5549 (2020).
3. Borczuk, A. C. et al. COVID-19 pulmonary pathology: a multi-institutional autopsy cohort from Italy and New York City. Mod Pathol, doi: 10.1038/s41379-020-00661-1 (2020).
4. Li, Y. et al. Progression to Fibrosing Diffuse Alveolar Damage in a Series of 30 Minimally Invasive Autopsies with COVID-19 Pneumonia in Wuhan, China. Histopathology, doi: 10.1111/his. 14249 (2020).
5. Roden, A. C. et al. The Spectrum of Histopathologic Findings in Lungs of Patients with Fatal COVID-19 Infection. Arch Pathol Lab Med, doi: 10.5858/arpa.2020-0491-SA (2020).
6. Hussman, J. P. Cellular and Molecular Pathways of COVID-19 and Potential Points of Therapeutic Intervention. Front Pharmacol 11, 1169, doi: 10.3389/fphar.2020.01169 (2020).
7. Bratic, A. & Larsson, N. G. The role of mitochondria in aging. J Clin Invest 123, 951-957, doi: 10.1172/JCI64125 (2013).
8. Carsana, L. et al. Pulmonary post-mortem findings in a series of COVID-19 cases from northern Italy: a two-centre descriptive study. Lancet Infect Dis 20, 1135-1140, doi: 10.1016/S1473-3099(20)30434-5 (2020).
9. Lakdawala, S. S. & Menachery, V. D. The search for a COVID-19 animal model. Science 368, 942-943, doi: 10.1126/science.abc6141 (2020).
10. Duan, F. et al. Modeling COVID-19 with Human Pluripotent Stem Cell-Derived Cells Reveals Synergistic Effects of Anti-inflammatory Macrophages with ACE2 Inhibition Against SARS-COV-2. Res Sq, doi: 10.21203/rs.3.rs-62758/v1 (2020).
11. Mulay, A. et al. SARS-COV-2 infection of primary human lung epithelium for COVID-19 modeling and drug discovery. bioRxiv, doi: 10.1101/2020.06.29.174623 (2020).
12. Salahudeen, A. A. et al. Progenitor identification and SARS-COV-2 infection in human distal lung organoids. Nature, doi: 10.1038/s41586-020-3014-1 (2020).
13. Hild, M. & Jaffe, A. B. Production of 3-D Airway Organoids From Primary Human Airway Basal Cells and Their Use in High-Throughput Screening. Curr Protoc Stem Cell Biol 37, IE 9 1-IE 9 15, doi: 10.1002/cpsc. 1 (2016).
14. Rock, J. R. et al. Basal cells as stem cells of the mouse trachea and human airway epithelium. Proc Natl Acad Sci USA 106, 12771-12775, doi: 10.1073/pnas.0906850106 (2009).
15. Tadokoro, T., Gao, X., Hong, C. C., Hotten, D. & Hogan, B. L. BMP signaling and cellular dynamics during regeneration of airway epithelium from basal progenitors. Development 143, 764-773, doi: 10.1242/dev.126656 (2016).
16. Suzuki, T. et al. Generation of human bronchial organoids for SARS-COV-2 research. BioRxiv, doi:https://doi.org/10.1101/2020.05.25.115600 (2020).
17. Mou, H. et al. Dual SMAD Signaling Inhibition Enables Long-Term Expansion of Diverse Epithelial Cells. Basal Cell Stem Cell 19, 217-231, doi: 10.1016/j.stem.2016.05.012 (2016).
18. Randell, S. H., Fulcher, M. L., O'Neal, W. & Olsen, J. C. Primary epithelial cell models for cystic fibrosis research. Methods Mol Biol 742, 285-310, doi: 10.1007/978-1-61779-120-8_18 (2011).
19. Wong, A. P. et al. Directed differentiation of human pluripotent stem cells into mature airway epithelia expressing functional CFTR protein. Nat Biotechnol 30, 876-882, doi: 10.1038/nbt.2328 (2012).
20. Ghaedi, M. et al. Human iPS cell-derived alveolar epithelium repopulates lung extracellular matrix. J Clin Invest 123, 4950-4962, doi: 10.1172/JCI68793 (2013).
21. Dye, B. R. et al. In vitro generation of human pluripotent stem cell derived lung organoids. Elife 4, doi: 10.7554/eLife.05098 (2015).
22. Konishi, S. et al. Directed Induction of Functional Multi-ciliated Cells in Proximal Airway Epithelial Spheroids from Human Pluripotent Stem Cells. Stem Cell Reports 6, 18-25, doi: 10.1016/j.stemcr.2015.11.010 (2016).
23. McCauley, K. B. et al. Efficient Derivation of Functional Human Airway Epithelium from Pluripotent Stem Cells via Temporal Regulation of Wnt Signaling. Cell Stem Cell 20, 844-857 e846, doi:10.1016/j.stem.2017.03.001 (2017).
24. Miller, A. J. et al. Generation of lung organoids from human pluripotent stem cells in vitro. Nat Protoc 14, 518-540, doi: 10.1038/s41596-018-0104-8 (2019).
25. Gotoh, S. et al. Generation of alveolar epithelial spheroids via isolated progenitor cells from human pluripotent stem cells. Stem Cell Reports 3, 394-403, doi:10.1016/j.stemcr.2014.07.005 (2014).
26. Jacob, A. et al. Differentiation of Human Pluripotent Stem Cells into Functional Lung Alveolar Epithelial Cells. Cell Stem Cell 21, 472-488 e410, doi: 10.1016/j.stem.2017.08.014 (2017).
27. Jacob, A. et al. Derivation of self-renewing lung alveolar epithelial type II cells from human pluripotent stem cells. Nat Protoc 14, 3303-3332, doi: 10.1038/s41596-019-0220-0 (2019).
28. Yamamoto, Y. et al. Long-term expansion of alveolar stem cells derived from human iPS cells in organoids. Nat Methods 14, 1097-1106, doi: 10.1038/nmeth.4448 (2017).
29. Chen, Y. W. et al. A three-dimensional model of human lung development and disease from pluripotent stem cells. Nat Cell Biol 19, 542-549, doi: 10.1038/ncb3510 (2017).
30. Huang, S. X. et al. Efficient generation of lung and airway epithelial cells from human pluripotent stem cells. Nat Biotechnol 32, 84-91, doi: 10.1038/nbt.2754 (2014).
31. Sachs, N. et al. Long-term expanding human airway organoids for disease modeling. EMBO J 38, doi: 10.15252/embj.2018100300 (2019).
32. van der Vaart, J. & Clevers, H. Airway organoids as models of human disease. J Intern Med, doi: 10.1111/joim. 13075 (2020).
33. Zhou, J. et al. Differentiated human airway organoids to assess infectivity of emerging influenza virus. Proc Natl Acad Sci USA 115, 6822-6827, doi: 10.1073/pnas. 1806308115 (2018).
34. Hoffmann, M. et al. SARS-COV-2 Cell Entry Depends on ACE2 and TMPRSS2 and Is Blocked by a Clinically Proven Protease Inhibitor. Cell 181, 271-280 e278, doi: 10.1016/j.cell.2020.02.052 (2020).
35. Zhao, Y. et al. Single-Cell RNA Expression Profiling of ACE2, the Receptor of SARS-COV-2. Am J Respir Crit Care Med 202, 756-759, doi: 10.1164/rccm.202001-0179LE (2020).
36. Jia, H. P. et al. ACE2 receptor expression and severe acute respiratory syndrome coronavirus infection depend on differentiation of human airway epithelia. J Virol 79, 14614-14621, doi: 10.1128/JVI.79.23.14614-14621.2005 (2005).
37. Hou, Y. J. et al. SARS-COV-2 Reverse Genetics Reveals a Variable Infection Gradient in the Respiratory Tract. Cell 182, 429-446 e414, doi: 10.1016/j.cell.2020.05.042 (2020).
38. Spagnolo, P. et al. Pulmonary fibrosis secondary to COVID-19: a call to arms? Lancet Respir Med 8, 750-752, doi: 10.1016/S2213-2600(20)30222-8 (2020).
39. Chen, Y. & Li, L. SARS-COV-2: virus dynamics and host response. Lancet Infect Dis 20, 515-516, doi: 10.1016/S1473-3099(20)30235-8 (2020).
40. Miyoshi, H. & Stappenbeck, T. S. In vitro expansion and genetic modification of gastrointestinal stem cells in spheroid culture. Nat Protoc 8, 2471-2482, doi: 10.1038/nprot.2013.153 (2013).
41. Sato, T. et al. Single Lgr5 stem cells build crypt-villus structures in vitro without a mesenchymal niche. Nature 459, 262-265, doi: 10.1038/nature07935 (2009).
42. Sayed, I. M. et al. Host engulfment pathway controls inflammation in inflammatory bowel disease. FEBS J, doi: 10.1111/febs.15236 (2020).
43. Giangreco, A., Reynolds, S. D. & Stripp, B. R. Terminal bronchioles harbor a unique airway stem cell population that localizes to the bronchoalveolar duct junction. Am J Pathol 161, 173-182, doi: 10.1016/S0002-9440(10)64169-7 (2002).
44. Kim, C. F. et al. Identification of bronchioalveolar stem cells in normal lung and lung cancer. Cell 121, 823-835, doi: 10.1016/j.cell.2005.03.032 (2005).
45. Engevik, M. A. et al. Human Clostridium difficile infection: inhibition of NHE3 and microbiota profile. Am J Physiol Gastrointest Liver Physiol 308, G497-509, doi: 10.1152/ajpgi.00090.2014 (2015).
46. Forbester, J. L. et al. Interaction of Salmonella enterica Serovar Typhimurium with Intestinal Organoids Derived from Human Induced Pluripotent Stem Cells. Infect Immun 83, 2926-2934, doi: 10.1128/IAI.00161-15 (2015).
47. Leslie, J. L. et al. Persistence and toxin production by Clostridium difficile within human intestinal organoids result in disruption of epithelial paracellular barrier function. Infect Immun 83, 138-145, doi: 10.1128/IAI.02561-14 (2015).
48. Williamson, I. A. et al. A High-Throughput Organoid Microinjection Platform to Study Gastrointestinal Microbiota and Luminal Physiology. Cell Mol Gastroenterol Hepatol 6, 301-319, doi: 10.1016/j.jcmgh.2018.05.004 (2018).
49. Porotto, M. et al. Authentic Modeling of Human Respiratory Virus Infection in Human Pluripotent Stem Cell-Derived Lung Organoids. mBio 10, doi: 10.1128/mBio.00723-19 (2019).
50. Han, Y. et al. Identification of Candidate COVID-19 Therapeutics using hPSC-derived Lung Organoids. bioRxiv, doi: 10.1101/2020.05.05.079095 (2020).
51. Huang, J. et al. SARS-COV-2 Infection of Pluripotent Stem Cell-Derived Human Lung Alveolar Type 2 Cells Elicits a Rapid Epithelial-Intrinsic Inflammatory Response. Cell Stem Cell, doi: 10.1016/j.stem.2020.09.013 (2020).
52. Han, Y. et al. Identification of SARS-COV-2 inhibitors using lung and colonic organoids. Nature, doi: 10.1038/s41586-020-2901-9 (2020).
53. Prytherch, Z. et al. Tissue-Specific stem cell differentiation in an in vitro airway model. Macromol Biosci 11, 1467-1477, doi: 10.1002/mabi.201100181 (2011).
54. Dvorak, A., Tilley, A. E., Shaykhiev, R., Wang, R. & Crystal, R. G. Do airway epithelium air-liquid cultures represent the in vivo airway epithelium transcriptome? Am J Respir Cell Mol Biol 44, 465-473, doi: 10.1165/rcmb.2009-0453OC (2011).
55. McGowan, S. E. The Formation of Pulmonary Alveoli. The Lung (Second Edition) Book Chapter (2014).
56. Liu, M., Gu, C. & Wang, Y. Upregulation of the tight junction protein occludin: effects on ventilation-induced lung injury and mechanisms of action. BMC Pulm Med 14, 94, doi: 10.1186/1471-2466-14-94 (2014).
57. Schlingmann, B., Molina, S. A. & Koval, M. Claudins: Gatekeepers of lung epithelial function. Semin Cell Dev Biol 42, 47-57, doi:10.1016/j.semcdb.2015.04.009 (2015).
58. Yang, J. et al. The development and plasticity of alveolar type 1 cells. Development 143, 54-65, doi: 10.1242/dev. 130005 (2016).
59. Rayner, R. E., Makena, P., Prasad, G. L. & Cormet-Boyaka, E. Optimization of Normal Human Bronchial Epithelial (NHBE) Cell 3D Cultures for in vitro Lung Model Studies. Sci Rep 9, 500, doi: 10.1038/s41598-018-36735-z (2019).
60 Shami, S. G. & Evans, M. J. Kinetics of Pulmonary Cells. In book: Comparative Biology of the Normal Lung, doi:DOI: 10.1016/B978-0-12-404577-4.15002-7 (2015).
61. Abdelwahab, E. M. M. et al. Wnt signaling regulates trans-differentiation of stem cell like type 2 alveolar epithelial cells to type 1 epithelial cells. Respir Res 20, 204, doi: 10.1186/s12931-019-1176-x (2019).
62. Gras, D. et al. Epithelial ciliated beating cells essential for ex vivo ALI culture growth. BMC Pulm Med 17, 80, doi: 10.1186/s12890-017-0423-5 (2017).
63. Khelloufi, M. K. et al. Spatiotemporal organization of cilia drives multiscale mucus swirls in model human bronchial epithelium. Sci Rep 8, 2447, doi: 10.1038/s41598-018-20882-4 (2018).
64. Kalsi, K. K., Sonya Jackson, S., Baines, D. L. Zonulin antagonist protects against tight junction disruption by Pseudomonas aeruginosa in airway epithelial cells. Faseb journal (Experimental Biology abstract), doi:https://doi.org/10.1096/fasebj.2020.34.s1.05094 (2020).
65. Knoops, K. et al. SARS-coronavirus replication is supported by a reticulovesicular network of modified endoplasmic reticulum. PLOS Biol 6, e226, doi:10.1371/journal.pbio.0060226 (2008).
66. Kaye, M. SARS-associated coronavirus replication in cell lines. Emerg Infect Dis 12, 128-133, doi: 10.3201/eid1201.050496 (2006).
67. Milewska, A. et al. Replication of Severe Acute Respiratory Syndrome Coronavirus 2 in Human Respiratory Epithelium. J Virol 94, doi: 10.1128/JVI.00957-20 (2020).
68. Zhu, N. et al. Morphogenesis and cytopathic effect of SARS-COV-2 infection in human airway epithelial cells. Nat Commun 11, 3910, doi: 10.1038/s41467-020-17796-z (2020).
69. Hui, K. P. Y. et al. Tropism, replication competence, and innate immune responses of the coronavirus SARS-COV-2 in human respiratory tract and conjunctiva: an analysis in ex-vivo and in-vitro cultures. Lancet Respir Med 8, 687-695, doi: 10.1016/S2213-2600(20)30193-4 (2020).
70. Rogers, T. F. et al. Isolation of potent SARS-COV-2 neutralizing antibodies and protection from disease in a small animal model. Science 369, 956-963, doi: 10.1126/science.abc7520 (2020).
71. Cox, R. M., Wolf, J. D. & Plemper, R. K. Therapeutically administered ribonucleoside analogue MK-4482/EIDD-2801 blocks SARS-COV-2 transmission in ferrets. Nat Microbiol, doi: 10.1038/s41564-020-00835-2 (2020).
72. Sheahan, T. P. et al. An orally bioavailable broad-spectrum antiviral inhibits SARS-COV-2 in human airway epithelial cell cultures and multiple coronaviruses in mice. Sci Transl Med 12, doi: 10.1126/scitranslmed.abb5883 (2020).
73. Nienhold, R. et al. Two distinct immunopathological profiles in autopsy lungs of COVID-19. MedRxiv, doi:https://doi.org/10.1101/2020.06.17.20133637 (2020).
74. Liao, M. et al. Single-cell landscape of bronchoalveolar immune cells in patients with COVID-19. Nat Med 26, 842-844, doi: 10.1038/s41591-020-0901-9 (2020).
75. Choi, J. et al. Inflammatory Signals Induce AT2 Cell-Derived Damage-Associated Transient Progenitors that Mediate Alveolar Regeneration. Cell Stem Cell 27, 366-382 e367, doi:10.1016/j.stem.2020.06.020 (2020).
76. Sahoo, D. et al. AI-guided discovery of the invariant host response to viral pandemics. EBiomedicine (Accepted) BioRxiv, doi:https://doi.org/10.1101/2020.09.21.305698 (2021).
77. Lamers, M. M. et al. An organoid-derived bronchioalveolar model for SARS-COV-2 infection of human alveolar type II-like cells. EMBO J 40, e105912, doi: 10.15252/embj.2020105912 (2021).
78. Nikolic, M. Z. & Rawlins, E. L. Lung Organoids and Their Use To Study Cell-Cell Interaction. Curr Pathobiol Rep 5, 223-231, doi: 10.1007/s40139-017-0137-7 (2017).
79. Bukys, M. A. et al. High-Dimensional Design-Of-Experiments Extracts Small-Molecule-Only Induction Conditions for Dorsal Pancreatic Endoderm from Pluripotency. iScience 23, 101346, doi: 10.1016/j.isci.2020.101346 (2020).
80. Sato, T. & Clevers, H. SnapShot: Growing Organoids from Stem Cells. Cell 161, 1700-1700 e1701, doi:10.1016/j.cell.2015.06.028 (2015).
81. Padela, S. et al. A critical role for fibroblast growth factor-7 during early alveolar formation in neonatal rat. Pediatr Res 63, 232-238, doi: 10.1203/PDR.0b013e31815f6e3a (2008).
82. Rabata, A., Fedr, R., Soucek, K., Hampl, A. & Koledova, Z. 3D Cell Culture Models Demonstrate a Role for FGF and WNT Signaling in Regulation of Lung Epithelial Cell Fate and Morphogenesis. Front Cell Dev Biol 8, 574, doi: 10.3389/fcell.2020.00574 (2020).
83. Yuan, T. et al. FGF10-FGFR2B Signaling Generates Basal Cells and Drives Alveolar Epithelial Regeneration by Bronchial Epithelial Stem Cells after Lung Injury. Stem Cell Reports 12, 1041-1055, doi:10.1016/j.stemcr.2019.04.003 (2019).
84. Yamamoto, K. et al. Type I alveolar epithelial cells mount innate immune responses during pneumococcal pneumonia. J Immunol 189, 2450-2459, doi: 10.4049/jimmunol. 1200634 (2012).
85. Wong, M. H. & Johnson, M. D. Differential response of primary alveolar type I and type II cells to LPS stimulation. PLOS One 8, e55545, doi: 10.1371/journal.pone.0055545 (2013).
86 Taubenberger, J. K. & Morens, D. M. The pathology of influenza virus infections. Annu Rev Pathol 3, 499-522, doi: 10.1146/annurev.pathmechdis.3.121806.154316 (2008).
87. Weinheimer, V. K. et al. Influenza A viruses target type II pneumocytes in the human lung. J Infect Dis 206, 1685-1694, doi: 10.1093/infdis/jis455 (2012).
88. Chan, R. W. et al. Tropism of and innate immune responses to the novel human betacoronavirus lineage C virus in human ex vivo respiratory organ cultures. J Virol 87, 6604-6614, doi: 10.1128/JVI.00009-13 (2013).
89. VanDussen, K. L., Sonnek, N. M. & Stappenbeck, T. S. L-WRN conditioned medium for gastrointestinal epithelial stem cell culture shows replicable batch-to-batch activity levels across multiple research teams. Stem Cell Res 37, 101430, doi: 10.1016/j.scr.2019.101430 (2019).
90. Ehaideb, S. N., Abdullah, M. L., Abuyassin, B. & Bouchama, A. A systematic review uncovers a wide-gap between COVID-19 in humans and animal models MedRxiv, doi:https://doi.org/10.1101/2020.07.15.20147041 (2020).
91. (CAP), C. o. A. P. Amended COVID-19 autopsy guideline statement from the CAP autopsy committee. (2020).
92. (CDC), C. f. D. C. a. P. Collection and Submission of Post-mortem Specimens from Deceased Persons with Known or Suspected COVID-19 (Interim Guidance). (2020).
93. Ghosh, P. et al. The stress polarity signaling (SPS) pathway serves as a marker and a target in the leaky gut barrier: implications in aging and cancer. Life Sci Alliance 3, doi: 10.26508/lsa.201900481 (2020).
94. Saved, I. M. et al. The DNA Glycosylase NEIL2 Suppresses Fusobacterium-Infection-Induced Inflammation and DNA Damage in Colonic Epithelial Cells. Cells 9, doi:doi: 10.3390/cells9091980 (2020).
95. Sayed, I. M. et al. Helicobacter pylori infection downregulates the DNA glycosylase NEIL2, resulting in increased genome damage and inflammation in gastric epithelial cells. J Biol Chem 295, 11082-11098, doi:10.1074/jbc.RA119.009981 (2020).
96. Corman, V. M. et al. Detection of 2019 novel coronavirus (2019-nCOV) by real-time RT-PCR. Euro Surveill 25, doi: 10.2807/1560-7917.ES.2020.25.3.2000045 (2020).
97. Lamers, M. M. et al. SARS-COV-2 productively infects human gut enterocytes. Science 369, 50-54, doi: 10.1126/science.abc1669 (2020).
98. Lopez-Sanchez, I. et al. GIV/Girdin is a central hub for profibrogenic signalling networks during liver fibrosis. Nat Commun 5, 4451, doi: 10.1038/ncomms5451 (2014).
99. Edgar, R., Domrachev, M. & Lash, A. E. Gene Expression Omnibus: NCBI gene expression and hybridization array data repository. Nucleic Acids Res 30, 207-210 (2002).
100. Barrett, T. et al. NCBI GEO: mining millions of expression profiles—database and tools. Nucleic Acids Res 33, D562-566, doi: 10.1093/nar/gki022 (2005).
101. Barrett, T. et al. NCBI GEO: archive for functional genomics data sets—update. Nucleic Acids Res 41, D991-995, doi: 10.1093/nar/gks1193 (2013).
102. Irizarry, R. A. et al. Summaries of Affymetrix GeneChip probe level data. Nucleic Acids Res 31, e15 (2003).
103. Irizarry, R. A. et al. Exploration, normalization, and summaries of high density oligonucleotide array probe level data. Biostatistics 4, 249-264, doi: 10.1093/biostatistics/4.2.249 (2003).
104. Li, B. & Dewey, C. N. RSEM: accurate transcript quantification from RNA-Seq data with or without a reference genome. BMC Bioinformatics 12, 323, doi:10.1186/1471-2105-12-323 (2011).
105. Pachter, L. Models for transcript quantification from RNA-Seq. arXiv e-prints (2011). <https://ui.adsabs.harvard.edu/#abs/2011arXiv1104.3889P>.
106. Dalerba, P. et al. Single-cell dissection of transcriptional heterogeneity in human colon tumors. Nat Biotechnol 29, 1120-1127, doi: 10.1038/nbt.2038 (2011).
107. Dalerba, P. et al. CDX2 as a Prognostic Biomarker in Stage II and Stage III Colon Cancer. N Engl J Med 374, 211-222, doi: 10.1056/NEJMoal 506597 (2016).
108. Volkmer, J. P. et al. Three differentiation states risk-stratify bladder cancer into distinct subtypes. Proc Natl Acad Sci USA 109, 2078-2083, doi: 10.1073/pnas. 1120605109 (2012).
109. Love, M. I., Huber, W. & Anders, S. Moderated estimation of fold change and dispersion for RNA-seq data with DESeq2. Genome Biol 15, 550, doi: 10.1186/s13059-014-0550-8 (2014).
110. Fabregat, A. et al. The Reactome Pathway Knowledgebase. Nucleic Acids Res 46, D649-D655, doi: 10.1093/nar/gkx1132 (2018).
111. Stuart, T. et al. Comprehensive Integration of Single-Cell Data. Cell 177, 1888-1902 e1821, doi: 10.1016/j.cell.2019.05.031 (2019).
112. Wolf, F. A., Angerer, P. & Theis, F. J. SCANPY: large-scale single-cell gene expression data analysis. Genome Biol 19, 15, doi: 10.1186/s13059-017-1382-0 (2018).
113. Zhang, Z. et al. SCINA: A Semi-Supervised Subtyping Algorithm of Single Cells and Bulk Samples. Genes (Basel) 10, doi: 10.3390/genes 10070531 (2019).
114. Bonser, L. R. et al. Flow-Cytometric Analysis and Purification of Airway Epithelial-Cell Subsets. Am J Respir Cell Mol Biol 64, 308-317, doi: 10.1165/rcmb.2020-0149MA (2021).
115. Miyoshi, H., and Stappenbeck, T. S. (2013). In vitro expansion and genetic modification of gastrointestinal stem cells in spheroid culture. Nat Protoc 8, 2471-2482.
116. Sachs, N., Papaspyropoulos, A., Zomer-van Ommen, D. D., Heo, I., Bottinger, L., Klay, D., Weeber, F., Huelsz-Prince, G., Iakobachvili, N., Amatngalim, G. D., et al. (2019). Long-term expanding human airway organoids for disease modeling. EMBO J 38.
117. Sato, T., Vries, R. G., Snippert, H. J., van de Wetering, M., Barker, N., Stange, D. E., van Es, J. H., Abo, A., Kujala, P., Peters, P. J., et al. (2009). Single Lgr5 stem cells build crypt-villus structures in vitro without a mesenchymal niche. Nature 459, 262-265.
118. Sayed, I. M., Suarez, K., Lim, E., Singh, S., Pereira, M., Ibeawuchi, S. R., Katkar, G., Dunkel, Y., Mittal, Y., Chattopadhyay, R., et al. (2020). Host engulfment pathway controls inflammation in inflammatory bowel disease. FEBS J.
119. Zhou, J., Li, C., Sachs, N., Chiu, M. C., Wong, B. H., Chu, H., Poon, V. K., Wang, D., Zhao, X., Wen, L., et al. (2018). Differentiated human airway organoids to assess infectivity of emerging influenza virus. Proceedings of the National Academy of Sciences of the United States of America 115, 6822-6827.
120. Tindle C, Fuller M, Fonseca A, Taheri S, Ibeawuchi S R, Beutler N, Katkar G D, Claire A, Castillo V, Hernandez M, Russo H, Duran J, Crotty Alexander L E, Tipps A, Lin G, Thistlethwaite P A, Chattopadhyay R, Rogers T F, Sahoo D, Ghosh P, Das S. Adult stem cell-derived complete lung organoid models emulate lung disease in COVID-19. Elife. 2021 Aug. 13; 10:e66417. doi: 10.7554/eLife.66417. PMID: 34463615

What is claimed is:

1. A method of modeling a biological process in a human lung, the method comprising:
providing a culture composition comprising human lung proximal airway epithelial cells and distal airway epithelial cells; and
infecting the culture composition with a respiratory pathogen.

2. The method of claim 1, wherein the culture composition is derived from a monolayer of stem cell-derived adult lung organoids (ALOs).

3. The method of claim 1, wherein the proximal airway epithelial cells are proximal ciliated cells.

4. The method of claim 1, wherein the distal airway epithelial cells are distal alveolar cells.

5. The method of claim 4, wherein at least a portion of the distal alveolar cells are differentiated to alveolar type-I (AT1) pneumocytes.

6. The method of claim 1, wherein the respiratory pathogen is SARS-COV-2, such that infecting the culture composition creates a lung model of COVID-19 disease.

7. The method of claim 6, further comprising before the infecting step, analyzing the lung model for presence of viral entry markers angiotensin-converting enzyme-II (ACE2) and Transmembrane Serine Protease 2 (TMPRSS2).

8. The method of claim 1, wherein the proximal airway epithelial cells permit viral infection.

9. The method of claim 5, wherein the distal alveolar cell differentiation permits viral propagation.

10. The method of claim 1, wherein the culture is grown in a conditioned media from L-WRN cells which express Wnt3, R-spondin and Noggin.

11. A method of identifying a therapeutic agent effective for treating a respiratory disease, the method comprising:
using the method of modeling a biological process in a human lung of claim 1;
administering the therapeutic agent to the cell culture composition before or after the infecting step; and
determining whether the therapeutic agent is effective in treating or preventing the respiratory disease caused by the infection in the lung model.

12. The method of claim 11, wherein the culture composition is derived from a monolayer of stem cell-derived adult lung organoids (ALOs).

13. The method of claim 11, wherein the proximal airway epithelial cells are proximal ciliated cells.

14. The method of claim 11, wherein the distal airway epithelial cells are distal alveolar cells.

15. The method of claim 14, wherein at least a portion of the distal alveolar cells are differentiated to alveolar type-I (AT1) pneumocytes.

16. The method of claim 11, wherein the respiratory pathogen is SARS-COV-2, such that infecting the culture composition creates an in vitro lung model of COVID-19 disease.

17. A human lung organoid model comprising:
a culture composition comprising human lung proximal airway epithelial cells and distal airway epithelial cells.

18. The model of claim 17, wherein the culture composition is derived from a monolayer of stem cell-derived adult lung organoids (ALOs).

19. The model of claim 17, wherein the proximal airway epithelial cells are proximal ciliated cells.

19. The model of claim 17, wherein the distal airway epithelial cells are distal alveolar cells.

20. The model of claim 17, wherein the model is an in vitro lung model of COVID-19 disease.

21. The model of claim 20, wherein at least a portion of the distal alveolar cells are differentiated to alveolar type-I (AT1) pneumocytes.
